(19) United States
(12) Patent Application Publication
(10) Pub. No.: US 2016/0331673 A1
(43) Pub. Date: Nov. 17, 2016

(54) COSMETIC COMPOSITION COMPRISING ELASTOMERS

(71) Applicants: DOW CORNING CORPORATION, Midland, MI (US); DOW CORNING (CHINA) HOLDING CO., LTD, Shanghai (CN)

(72) Inventors: Michael Salvatore FERRITTO, Midland, MI (US); Donald Anthony KADLEC, Midland, MI (US); Kimmai Thi NGUYEN, Midland, MI (US); Paul W. PRETZER, Midland, MI (US); Justin M. SCHERZER, Freeland, MI (US); Adriana C. URRUTIA, Mexico City (MX); Isabelle VAN REETH, Shanghai (CN); Kenneth ZIMMERMAN, Midland, MI (US)

(73) Assignees: Dow Coming Corporation, Midland, MI (US); Dow Coming (China) Holding Co., Ltd., Shanghai (CN)

(21) Appl. No.: 15/112,514

(22) PCT Filed: Jan. 16, 2015
(86) PCT No.: PCT/CN2015/070857

§ 371 Date: Jul. 19, 2016

(30) Foreign Application Priority Data
Jan. 29, 2014 (CN) 201410042747.3

(51) Int. Cl. A61K 8/898; A61Q 1/04; A61Q 17/04; A61Q 5/06; A61Q 19/00; A61Q 5/02; A61Q 5/12; A61Q 1/02; A61Q 1/06; A61Q 15/00

(52) U.S. Cl. CPC A61K 8/898; A61Q 1/06; A61Q 1/04; A61Q 17/04; A61Q 15/00; A61Q 19/00; A61Q 5/02; A61Q 5/12; A61Q 1/02; A61Q 5/06; A61K 2800/42; A61K 2800/592; A61K 2800/10

(57) ABSTRACT

A cosmetic composition comprises a silicone organic elastomer comprising an amino functional group, and at least one cosmetic ingredient, generally in a cosmetically acceptable medium. The silicone organic elastomer is a reaction product of: (A) a linear, branched or cyclic organohydrogensiloxane comprising at least one (1) silicon-bonded hydrogen atom; and (B) a XZ′n derivative comprising at least two (2) unsaturated aliphatic groups; in the presence of (C) a hydrosilylation catalyst. X is an amine group containing compound, Z′ is a ring-opened ethylenically-unsaturated epoxide comprising at least one (1) unsaturated aliphatic group and n=1 or 2. A process of preparing the cosmetic composition and method of using the cosmetic composition are also disclosed.

### CROSS REFERENCE

This application claims priority on China national application No. 201410042747.3, filed on Jan. 29, 2014.

### TECHNICAL FIELD

This disclosure relates to cosmetic compositions comprising a silicone organic elastomer comprising an amino functional group, and at least one cosmetic ingredient, optionally in a cosmetically acceptable medium.

The silicone organic elastomer is a reaction product of a linear, branched or cyclic organohydrogensiloxane (A) comprising at least 1 silicon-bonded hydrogen atom, and a XZ′ₙ derivative (B) comprising at least 2 unsaturated aliphatic groups, where X is an amine group containing compound, Z′ is a ring-opened ethylenically-unsaturated epoxide comprising at least 1 unsaturated aliphatic group and n=1 or 2, and (C) a hydrosilylation catalyst.

The silicone organic elastomer comprising an amino functional group is particularly substantive to keratinous substrates, such as skin and hair.

### BACKGROUND OF THE INVENTION

Silicone elastomer gels have been used extensively to enhance the aesthetics of personal care formulations by providing a unique sensory profile upon application. Most silicone elastomer gels are obtained by a crosslinking hydrosilylation reaction of an SiH polysiloxane with another polysiloxane containing an unsaturated hydrocarbon substituent, such as a vinyl functional polysiloxane, or by crosslinking an SiH polysiloxane with a hydrocarbon diene or with a terminally unsaturated polyoxyalkylene. There have been many attempts to improve compatibilities of silicone elastomers with various personal care ingredients wherein alkyls, polyether, amines or other organofunctional groups have been grafted onto the silicone organic elastomer backbone. Silicone elastomers may be formed in the presence of a carrier fluid, such as a volatile silicone or organic fluid, resulting in a gel composition. The silicone elastomer may be formed at higher solids content, subsequently sheared and admixed with additional carrier fluid to also create gels paste compositions.

Silicone elastomer gels have a variety of uses in personal and health care compositions where they may provide for sensory characteristics, such as velvety or powdery feel. They also find application in hair care, such as in hair colouring products.

Some elastomers also have emulsifying properties.

However, there is still a need to improve the substantivity of silicone elastomer gels to keratinous substrates without sacrificing sensory aesthetic profiles. Furthermore, the gelling or thickening efficiency of the silicone elastomer in a carrier fluid should be maintained or improved.

The present inventors have discovered silicone organic elastomers comprising an amino functional group based on certain crosslinkers such as a XZ′ₙ derivative comprising at least 2 unsaturated aliphatic groups, provide compositions with improved substantivity on keratinous substrates, while maintaining sensory aesthetics.

### BRIEF SUMMARY OF THE INVENTION

The present invention relates to cosmetic compositions comprising a silicone organic elastomer comprising an amino functional group and at least one cosmetic ingredient, optionally in a cosmetically acceptable medium.

The silicone organic elastomer is a reaction product of a linear, branched or cyclic organohydrogensiloxane (A) comprising at least 1 silicon-bonded hydrogen atom, and a XZ′ₙ derivative (B) comprising at least 2 unsaturated aliphatic groups, where X is an amine group containing compound, Z′ is a ring-opened ethylenically-unsaturated epoxide comprising at least 1 unsaturated aliphatic group and n=1 or 2, and (C) a hydrosilylation catalyst.

The invention also relates to a process to prepare the cosmetic compositions and uses of said cosmetic compositions.

### DETAILED DESCRIPTION OF THE INVENTION

The present invention relates to cosmetic compositions comprising a silicone organic elastomer comprising an amino functional group and at least one cosmetic ingredient, optionally in a cosmetically acceptable medium.

The silicone organic elastomer is a reaction product of a linear, branched or cyclic organohydrogensiloxane (A) comprising at least 1 silicon-bonded hydrogen atom, and a XZ′ₙ derivative (B) comprising at least 2 unsaturated aliphatic groups, where X is an amine group containing compound, Z′ is a ring-opened ethylenically-unsaturated epoxide comprising at least 1 unsaturated aliphatic group and n=1 or 2, and (C) a hydrosilylation catalyst.

The silicone organic elastomer comprising an amino functional group comprises an amino functional group grafted to the organopolysiloxane via a hydrosilylation reaction between

A. an organohydrogensiloxane comprising at least 1 silicon-bonded hydrogen atom, and
B. a XZ′ₙ derivative (B) comprising at least 2 unsaturated aliphatic groups

i. where X is an amine group containing compound

ii. and Z′ is a ring-opened ethylenically unsaturated epoxide

iii. n=1 or 2

C. in the presence of a hydrosilylation catalyst (C)
D. in the presence of a carrier fluid (ii).

The organohydrogensiloxane comprising siloxy units may be represented by the average formula

(R¹₃SiO_{0.5})ᵥ(R²₂SiO)ₓ(R²HSiO)_{y}

wherein R¹ is hydrogen or R²,
R² is a monovalent hydrocarbyl
v≧2, x≧0, y≧1.

The silicone organic elastomer comprising an amino functional group may be provided in the form of a gel or paste, containing i) a silicone organic elastomer and ii) a carrier fluid. The gel and paste compositions are useful in personal care compositions.

The silicone organic elastomer is a highly crosslinked system.

### i) The Silicone Organic Elastomer

The silicone organic elastomers are obtainable as hydrosilylation reaction products of (A) an organohydrogensiloxane, (B) a XZ′ₙ derivative comprising at least 2 unsaturated aliphatic groups, and (C) a hydrosilylation catalyst.

The term “hydrosilylation” means the addition of an organosilicon compound containing silicon-bonded hydrogen, (such as component (A)) to a compound containing aliphatic unsaturated aliphatic group (such as component (B)), in the presence of a catalyst (such as component (C)). Hydrosilylation reactions are known in the art, and any such known methods or techniques may be used to effect the hydrosilylation reaction of components (A), (B), and (C) to prepare the silicone organic elastomers i).

The silicone organic elastomer may contain pendant, non-crosslinking groups, independently selected from hydrocarbon groups containing 2-30 carbons, polyoxyalkylene groups, XZ′ₙ derivatives containing one unsaturated aliphatic group, linear or branched siloxane polymer comprising one unsaturated aliphatic group, polyol component comprising one unsaturated aliphatic group and mixtures thereof. Such pendant groups result from the optional addition of a component (D), selected from component (D¹) a hydrocarbon containing 2-30 carbons having one terminal unsaturated aliphatic group, and/or component (D²) a polyoxyalkylene having one terminal unsaturated aliphatic group and/or component (D³) a XZ′ₙ derivative comprising one unsaturated aliphatic group and/or component (D⁴) a linear or branched siloxane polymer comprising one unsaturated aliphatic group, and/or component (D⁵) a polyol component comprising one unsaturated aliphatic group, to the silicone organic elastomer via a hydrosilylation reaction.

The hydrosilylation reaction to prepare the silicone organic elastomer may be conducted in the presence of a solvent, and the solvent subsequently removed by known techniques. Alternatively, the hydrosilylation may be conducted in a solvent, where the solvent is the same as the carrier fluid described as component ii).

### (A) The Organohydrogensiloxane

Organopolysiloxanes are polymers containing siloxy units independently selected from (R⁰₃SiO_{0.5}), (R⁰₂SiO), (R⁰SiO_{1.5}), or (SiO₂) siloxy units, where R⁰ may be any organic group. When R⁰ is a methyl group in the (R⁰₃SiO_{0.5}), (R⁰₂SiO), (R⁰SiO_{1.5}), or (SiO₂) siloxy units of an organopolysiloxane, the siloxy units are commonly referred to as M, D, T, and Q units respectively. These siloxy units can be combined in various manners to form cyclic, linear, or branched structures. The chemical and physical properties of the resulting polymeric structures can vary. For example organopolysiloxanes can be volatile or low viscosity fluids, high viscosity fluids, gums, elastomers or rubbers, and resins.

Organohydrogensiloxanes are organopolysiloxanes containing at least one silicon-bonded hydrogen atom (SiH), that is at least one siloxy unit in the organopolysiloxane has the formula (R⁰₂HSiO_{0.5}), (R⁰HSiO), or (HSiO_{1.5}). These siloxy units can be represented as M^{H}, D^{H}, and T^{H} siloxy units respectively when R⁰ is methyl.

Component (A) of the present invention is an organohydrogensiloxane having an average, per molecule, of at least one SiH units. The average of SiH units on the organohydrogensiloxane may range of from 1 to 1000, alternatively, of from 1 to 500, alternatively of from 1 to 250.

The organohydrogensiloxanes useful in the present invention may be cyclic, linear or branched, and comprise any number of (R⁰₃SiO_{0.5}), (R⁰₂SiO), (R⁰SiO_{1.5}), (R⁰₂HSiO_{0.5}), (R⁰HSiO), (HSiO_{1.5}) or (SiO₂) siloxy units, providing there are on average at least two SiH siloxy units in the molecule.

Component (A) can be a single linear or branched organohydrogensiloxane or a combination comprising two or more linear or branched organohydrogensiloxanes that differ in at least one of the following properties: structure, viscosity, average molecular weight, siloxy units, and sequence.

The organohydrogensiloxane may have the average formula

(R¹₃SiO_{0.5})ᵥ(R²₂SiO)ₓ(R²HSiO)_{y}

wherein
R¹ is hydrogen or R²,
R² is a monovalent organic group,
v≧2,
x≧0, alternatively x=1 to 500, alternatively x=1 to 200,
y≧1, alternatively y=2 to 200, alternatively y=2 to 100.

The monovalent organic group R² may be an aliphatic hydrocarbyl, an aromatic hydrocarbyl, or an organyl group (that is any organic substituent group, regardless of functional type, having one free valence at a carbon atom). Aliphatic hydrocarbyls are exemplified by, but not limited to alkyl groups such as methyl, ethyl, propyl, pentyl, octyl, undecyl, and octadecyl and cycloalkyl groups such as cyclohexyl. Aromatic hydrocarbyl groups are exemplified by, but not limited to, phenyl, tolyl, xylyl, benzyl, styryl, and 2-phenylethyl. Organyl groups are exemplified by, but not limited to, halogenated alkyl groups such as chloromethyl, 3-chloropropyl, and 3,3,3-trifluoropropyl; nitrogen containing groups such as amino groups, amido groups, imino groups, imido groups; oxygen containing groups such as polyoxyalkylene groups, carbonyl groups. Further organyl groups may include sulfur containing groups, fluor containing groups, phosphorus containing groups, boron containing groups.

The organohydrogensiloxane may contain additional siloxy units and have the average formula

(R¹₃SiO_{0.5})ᵥ(R²₂SiO)ₓ(R²HSiO)_{y}(R²SiO_{1.5})_{z},

(R¹₃SiO_{0.5})ᵥ(R²₂SiO)ₓ(R²FHSiO)_{y}(SiO₂)_{w},

(R¹₃SiO_{0.5})ᵥ(R²₂SiO)ₓ(R²FHSiO)_{y}(SiO₂)_{w}(R²SiO_{1.5})_{z}

or any mixture thereof,
where
R¹ is hydrogen or R²,
R² is a monovalent organic group,
and v≧2, w≧0, x≧0, 1, and z is 0.

The organohydrogensiloxane may be selected from a dimethyl, methyl-hydrogen polysiloxane having the average formula;

(CH₃)₃SiO[(CH₃)₂SiO]ₓ[(CH₃)HSiO]_{y}Si(CH₃)₃

where x≧0, alternatively, x=1 to 500, alternatively x=1 to 200,
and y≧1, alternatively, y=2 to 200, alternatively y=2 to 100.

The organohydrogensiloxane may be a mixture of dimethyl, methyl-hydrogen polysiloxane having the average formula (CH₃)₃SiO[(CH₃)₂SiO]ₓ[(CH₃)HSiO]_{y}Si(CH₃)₃ and SiH terminal dimethyl polysiloxane having the average formula H(CH₃)₂SiO[(CH₃)₂SiO]ₓSi(CH₃)₂H where x and y are as defined above. The amount of each organohydrogensiloxane in the mixture may vary, or alternatively may be such that in the mixture 0 to 85 wt %, alternatively 10 to 70 wt %, alternatively 20 to 60 wt % or alternatively 30 to 50 wt % of the total SiH in the mixture is from the SiH content of the SiH terminal dimethyl polysiloxane.

The organohydrogensiloxane may have the average formula

H(CH₃)₂SiO[(CH₃)₂SiO]ₓ[(CH₃)HSiO]_{y}Si(CH₃)₂H

where x and y are as defined above.

The organohydrogensiloxane having at least two SiH may further be an organohydrogencyclosiloxane having the formula [R²HSiO]_{g} where R² is a is a monovalent organic group and g≧3.

The organohydrogensiloxane having at least two SiH may further be an organohydrogensiloxane which contains cyclosiloxane rings in its molecule, each ring having at least one silicon bonded hydrogen (SiH) unit.

Cyclosiloxane rings contain at least three siloxy units (that is the minimum needed in order to form a siloxane ring), and may be any combination of (R₃SiO_{0.5}), (R₂SiO), (RSiO_{1.5}), or (SiO₂) siloxy units that forms a cyclic structure, providing at least one of the cyclic siloxy units on each siloxane ring contains one SiH unit, that is there is at least one (R₂HSiO_{0.5}), (RHSiO), or a (HSiO_{1.5}) siloxy unit present in the ring.

The cyclosiloxane rings of the organohydrogensiloxane are linked together by a divalent organic or siloxane group, or combination thereof. The divalent linking group may be designated as Y and the cyclosiloxane as G. Thus, the organohydrogensiloxane of the present invention may be represented by the general formula G-[Y-G]ₐ, where G is a cyclosiloxane as described above and Y is a divalent organic, a siloxane, a polyoxyalkylene group, or combination thereof, and the subscript a is greater than zero.

When Y is a divalent organic, it may be a divalent hydrocarbon containing 1 to 30 carbons, either as aliphatic or aromatic structures, and may be branched or unbranched. Alternatively, Y can be an alkylene group containing 2 to 20 carbons, or alternatively containing 4 to 12 carbons.

When Y is a divalent organic, it may also be selected from an organic polymer, such as a polyoxyalkylene group.

When Y is a siloxane group it may be selected from any organopolysiloxane containing at least two divalent hydrocarbon groups, designated as R¹. Thus, the siloxane linking group can be any organopolysiloxane comprising at least two siloxane units represented by the average formula R¹RₘSiO_{(3-m)/2} wherein R is an organic group, R¹ is a divalent hydrocarbon, and 0≦m≦3.

The R¹ group may be present on any mono, di, or tri-siloxy unit in an organopolysiloxane molecule, for example; (R¹R₂SiO_{0.5}), (R¹RSiO), or (R¹SiO_{1.5}), as well as in combination with other siloxy units not containing an R¹ substituent, such as (R₃SiO_{0.5}), (R₂SiO), (RSiO_{1.5}), or (SiO₂) siloxy units where R is independently any organic group providing there are at least two R¹ substituents in the organopolysiloxane. Representative R¹ groups include; ethylene, propylene, butylene, isobutylene, hexylene, and similar homologs. Alternatively, R¹ is ethylene.

Representative, non-limiting, examples of such siloxane based structures suitable as siloxane linking groups include;

(R₂R¹SiO_{0.5})(R₂SiO)ₓ(R₂R¹SiO_{0.5});

(R³SiO_{0.5})(R₂SiO)ₓ(R¹RSiO)_{y}(R₃SiO_{0.5});

(R³SiO_{0.5})(R₂SiO)ₓ(R¹RSiO)_{y}(RSiO_{1.5}),(R₃SiO_{0.5});

where x≧0, y≧2, and z≧0.

Organohydrogensiloxanes having at least two SiH containing cyclosiloxane rings in its molecule may be selected from any of the organohydrogensiloxanes taught in WO03/093349, which is herein incorporated by reference for its teaching of suitable organohydrogensiloxanes.

The organohydrogensiloxanes having at least two SiH containing cyclosiloxane rings in its molecule typically have a viscosity from 5 to 50,000 mPa·s, alternatively from 10 to 10,000 mPa·s, or alternatively from 25 to 2,000 mPa·s.

Representative, non-limiting examples of organohydrogensiloxanes having at least two SiH containing cyclosiloxane rings in its molecule include:

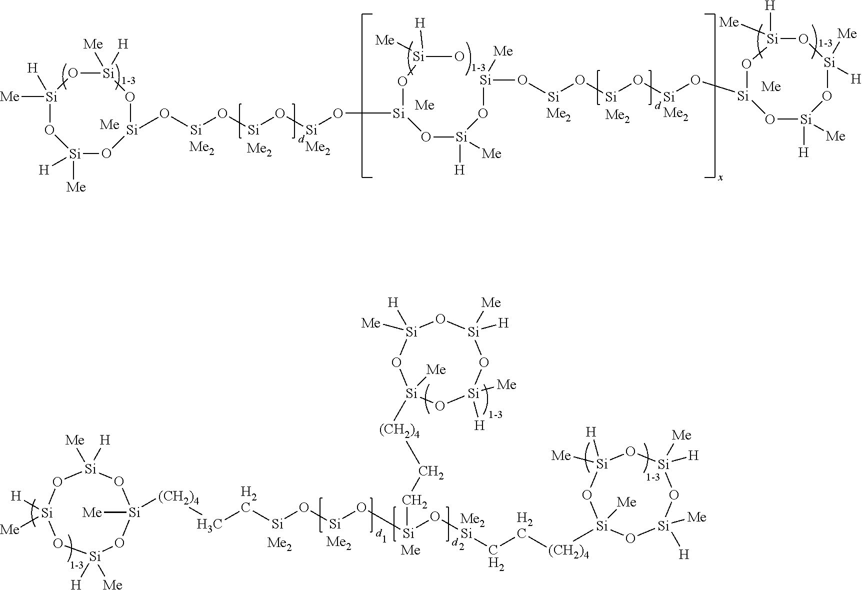

Methods for preparing organohydrogensiloxanes are well known in the art, and many are sold commercially.

### (B) the XZ′ₙ Derivative B Comprising at Least 2 Unsaturated Aliphatic Groups

The XZ′ₙ derivative (B) comprising at least 2 unsaturated aliphatic groups is the reaction product of an amine group containing compound X and at least one ethylenically-unsaturated epoxide Z comprising at least 1 unsaturated aliphatic group and n=1 or 2, reaction upon which the epoxide of ethylenically-unsaturated epoxide Z is opened to produce the ring opened ethylenically-unsaturated epoxide Z′.

The ethylenically-unsaturated epoxide Z contains (#1) the oxirane ring/epoxy group which provides for reaction with the amine group containing compound X, and (#2) the at least 1 unsaturated aliphatic group (or unsaturated group) which provides for reaction with organohydrogensiloxane (A).

The average of unsaturated aliphatic groups on the XZ′ₙ derivative (B) may range of from 2 to 30, alternatively of from 2 to 10, alternatively of from 2 to 5.

The amine group containing compound X is exemplified by primary amines, secondary amines or tertiary amines.

The amine group containing compound X may be an aliphatic or aromatic primary or secondary amine, where the substituent(s) replacing the hydrogen atom(s) on the nitrogen may be selected from alkyl group containing from 1 to 30 carbon atoms, alcohols, ethers, aryl group, allyl groups.

The amine group containing compound X may be a proteinogenic or non-proteinogenic amino acid where the carboxylic acid function is inactivated. The carboxylic acid function may hinder reaction with component A) if active. Inactivation of the carboxylic acid function may be carried out as known in the art for the addition of protecting groups to carboxylic acid functions, such as esterification.

Non-limiting examples of primary amines include alkylamines (such as propylamine, hexadecylamine, octadecylamine); fatty amines (such as coco amine, tallow amine, soya amine, stearyl amine, rape oil amine); primary hetero cycloalkylamines (such as cyclopentylamine, cyclohexylamine); allylamines; aromatic amines (aniline, toluidine); diamines; polyamines; and derivatives or mixtures thereof.

Non-limiting examples of secondary amine include di-alkylamines (such as diisopropylamine, bis(1-methyl)propylamine, di-2-ethylhexylamine); secondary cycloalkylamines (such as N-ethylcyclohexylamine, dicyclohexylamine); hetero cyclic amines (such as pyrrolidine, piperidine, hexamethyleneimine, morpholine, piperazine); di-allyl amines; secondary aromatic amines (such as diarylamines, for example diphenylamine); and derivatives or mixtures thereof.

Non-limiting examples of tertiary amine include tertiary amines derived from fatty alcohols.

The amine group needs to be available for reaction with the epoxy group of component Z. The amine may thus be in terminal or in pendant position; typically, in terminal position.

The ethylenically unsaturated epoxide Z contains at least 1 epoxy group and at least one unsaturated aliphatic group in terminal position.

The ethylenically unsaturated epoxide Z has the structure (I):

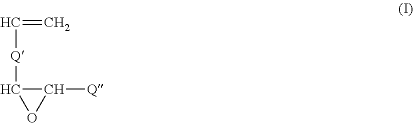

where Q′ is an organic group having 1 to 12 carbon atoms and is optionally present and Q″ is hydrogen or an organic group having 1 to 12 carbon atoms. In some instances, Q′ and Q″ may be substituted hydrocarbyl groups, containing a non carbon atom such as oxygen, phosphorus, halogen, nitrogen and/or sulfur.

Examples of ethylenically unsaturated epoxides include unsaturated glycidyl ethers, monoepoxides of dienes or polyenes, ethylenically unsaturated glycidyl esters, epoxy functional allyl polyether, etc.

Ethylenically unsaturated epoxides include butadiene mono epoxide, where Q′ is absent and Q″ is hydrogen; 1,2-epoxy-7-octene; methyl vinyl glycidyl amine; vinyl-3,4-epoxy cyclohexane; allyl-3,4-epoxy cyclohexane.

The unsaturated glycidyl ethers have the general formula (II):

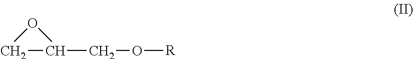

where R is an ethylenically unsaturated radical, as for example, ethylenically unsaturated aliphatic radicals such as vinyl, isopropenyl, allyl, methallyl, butenyl, oleyl, etc. and cycloalkyl or aryl radicals containing an ethylenically unsaturated substituent, when the ethylenically unsaturated substituent is not in a ring position, such as 4-vinylcyclohexyl, o-allylphenyl, p-vinyl benzyl, etc. R may also contain a non carbon atom such as oxygen, phosphorus, halogen, nitrogen and/or sulfur.

Exemplary of these ethers are vinyl glycidyl ether, allyl glycidyl ether, vinylcyclohexyl glycidyl ether, o-allylphenyl glycidyl ether, butenyl glycidyl ether, 2,3-epoxypropyl 4-vinyl phenyl ether, etc.

The monoepoxides of dienes and polyenes have the general formula (III):

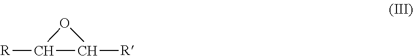

where R is an ethylenically unsaturated radical as defined above and R′ is hydrogen, R, alkyl, cycloalkyl, aryl or alkaryl, or R and R′ together with the two carbons of the epoxy group may form a cycloaliphatic ring which may be substituted by an ethylenically unsaturated hydrocarbon group, such as a vinyl group. Exemplary of the monoepoxides of dienes and polyenes are butadiene monoxide, 3,4-epoxy-1-pentene, 4,5-epoxy-2-pentene, 5,6-epoxy-2-hexene, 3,4-epoxy-1-vinylcyclohexene, 5,6-epoxy-1,7-octadiene, etc.

Another class of ethylenically unsaturated epoxides are the glycidyl esters of ethylenically unsaturated carboxylic acids which have the general formula (IV):

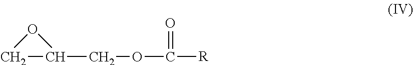

where R is an ethylenically unsaturated radical as described above.

Exemplary of such glycidyl esters are glycidyl acrylate, glycidyl methacrylate, glycidyl sorbate, glycidyl linoleate, glycidyl oleate, glycidyl 3-butenoate, glycidyl undecylenate; 2,3-epoxycinnamyl acrylate; 9,10-epoxyoleylacrylate; 2,3-epoxybutyl methacrylate; 3,4-epoxy-cyclohexyl acrylate.

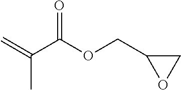

The ethylenically-unsaturated epoxide may be an epoxy functional allyl polyether having the general formula (V) or (VI):

CH₂═CH—(CH₂)ₐ—O—(C_{b}H_{2b}O)_{c}—(CH₂)ₙ—CH(—O—)—CH—R″ (V):

where R″ is hydrogen or an organic group having 1 to 30 carbon atoms, a is an integer in the range of from 1 to 30, b is an integer in the range of from 1 to 20, c is an integer in the range of from 0 to 50, n is an integer in the range of from 1 to 30;

CH₂═CH—(CH₂)ₐ—O—(CH₂CH(CH₃)O)_{d}—(CH₂CH₂O)ₑ—(CH₂)ₙ—CH(—O—)—CH—R″ (VI):

where n and a are defined as above, d is an integer in the range of from 1 to 20, e is an integer in the range of from 0 to 20.

The ethylenically-unsaturated epoxide may be an epoxy functional allyl polyether having the general formula (VII):

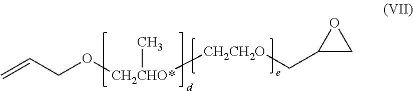

where d and e are as described above.

Different methods exist to produce the XZ′ₙ derivative comprising at least 2 unsaturated aliphatic groups (B), as the reaction product of an amine group containing compound X and at least one ethylenically-unsaturated epoxide Z, which methods are known in the art.

Such a method comprises mixing the amine group containing compound X and the at least one ethylenically-unsaturated epoxide Z, optionally in a solvent, optionally heating up to 120° C., over a time ranging of from 10 minutes to 24 hours, subsequently removing the optional solvent. The optional solvent may be the same or different from carrier fluid ii) discussed hereafter.

Primary amines react with epoxides to give a mixture of mono- and dioxyalkylated derivatives, whereas secondary amines give monooxyalkylated compounds, and tertiary amines form quaternary ammonium compounds.

Primary amines may thus react with 2 ethylenically-unsaturated epoxides containing each at least one unsaturated aliphatic group. For example, an alkylamine may react with 2 allyl glycidyl ether, providing for a XZ′₂ derivative comprising 2 unsaturated aliphatic groups.

Secondary amines may react with one ethylenically-unsaturated epoxide containing at least 2 unsaturated aliphatic groups. For example, a dialkylamine may react with one ethylenically unsaturated epoxide Z of formula

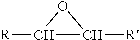

where R and R′ are ethylenically unsaturated radicals, providing for a XZ′ derivative comprising 2 unsaturated aliphatic groups.

Combinations may be numerous; provided there are at least 2 unsaturated aliphatic groups on the XZ′ₙ derivative to provide for the crosslinking function of component (B).

The component (B) may be used in conjunction with another crosslinker (B1), such as alpha, omega-diene; polyoxyalkylene comprising 2 unsaturated aliphatic groups; glycerol ethers comprising 2 unsaturated aliphatic groups; siloxane polymers comprising 2 unsaturated aliphatic groups. Such crosslinkers (B1) are well known in the art for forming silicone elastomers. Where a second crosslinker is used, the ratio of (B) and (B1) may range of from 1:10 to 10:1, alternatively 1:3 to 3:1.

The amounts of components (A) and (B) used in the hydrosilylation reaction may vary. Typically, the molar ratio of the SiH units of component (A) to the unsaturated groups of component (B) ranges of from 10/1 to 1/10, alternatively of from 5/1 to 1/5, or alternatively of from 2/1 to 1/2. In one embodiment, the molar ratio of the unsaturated groups in (B) to the SiH units in (A) is greater than 1.

### (C) The Hydrosilylation Catalyst

Component (C) comprises any catalyst typically employed for hydrosilylation reactions. It is preferred to use platinum group metal-containing catalysts. By platinum group it is meant ruthenium, rhodium, palladium, osmium, iridium and platinum and complexes thereof. Platinum group metal-containing catalysts useful in preparing the compositions of the present invention are the platinum complexes prepared as described by Willing, U.S. Pat. No. 3,419,593, and Brown et al, U.S. Pat. No. 5,175,325, each of which is hereby incorporated by reference to show such complexes and their preparation. Other examples of useful platinum group metal-containing catalysts can be found in Lee et al., U.S. Pat. No. 3,989,668; Chang et al., U.S. Pat. No. 5,036,117; Ashby, U.S. Pat. No. 3,159,601; Lamoreaux, U.S. Pat. No. 3,220,972; Chalk et al., U.S. Pat. No. 3,296,291; Modic, U.S. Pat. No. 3,516,946; Karstedt, U.S. Pat. No. 3,814,730; and Chandra et al., U.S. Pat. No. 3,928,629 all of which are hereby incorporated by reference to show useful platinum group metal-containing catalysts and methods for their preparation. The platinum group-containing catalyst can be platinum group metal, platinum group metal deposited on a carrier such as silica gel or powdered charcoal, or a compound or complex of a platinum group metal. Preferred platinum-containing catalysts include chloroplatinic acid, either in hexahydrate form or anhydrous form, and or a platinum-containing catalyst which is obtained by a method comprising reacting chloroplatinic acid with an aliphatically unsaturated organosilicon compound such as divinyltetramethyldisiloxane, or alkene-platinum-silyl complexes as described in U.S. patent application Ser. No. 10/017,229, filed Dec. 7, 2001, such as (COD)Pt(SiMeCl₂)₂, where COD is 1,5-cyclooctadiene and Me is methyl. These alkene-platinum-silyl complexes may be prepared, for example by mixing 0.015 mole (COD)PtCl₂ with 0.045 mole COD and 0.0612 moles HMeSiCl₂.

The appropriate amount of the catalyst will depend upon the particular catalyst used. The platinum catalyst should be present in an amount sufficient to provide at least 2 parts per million (ppm), alternatively 4 to 200 ppm of platinum based on total weight percent solids (all non-solvent ingredients) in the composition. Typically, the platinum is present in an amount sufficient to provide 4 to 150 weight ppm of platinum on the same basis. The catalyst may be added as a single species or as a mixture of two or more different species.

### (D) Optional Components Containing One Terminal Unsaturated Aliphatic Hydrocarbon Group

The silicone organic elastomer may also contain pendant, non-crosslinking moieties. These groups are formed on the silicone organic elastomer via a hydrosilylation reaction by the addition of component (D) a compound having one unsaturated aliphatic hydrocarbon group. Component (D) may be selected from (D¹) a hydrocarbon containing 6-30 carbons having one unsaturated aliphatic group, where the unsaturated group may be terminal, and/or component (D²) a polyoxyalkylene having one unsaturated aliphatic group where the unsaturated group may be terminal, and/or component (D³) a XZ′ₙ derivative having one unsaturated aliphatic group, and/or component (D⁴) a linear or branched siloxane polymer comprising one unsaturated aliphatic group, and/or component (D⁵) a polyol component comprising one unsaturated aliphatic group, or mixtures thereof.

The addition of component (D) can alter the resulting chemical and physical properties of the silicone organic elastomer. For example, selecting (D¹) will result in the addition of hydrocarbon groups to the silicone organic elastomer, thus adding more hydrophobic character to the silicone organic elastomer. Conversely, selecting a polyoxyalkylene having a majority of ethylene oxide units will result in a silicone organic elastomer having increased hydrophilicity, which can subsequently incorporate water or hydrophilic components with the silicone organic elastomer to form dispersions or pastes.

The unsaturated aliphatic hydrocarbon group in (D) can be an alkenyl or alkynyl group. Representative, non-limiting examples of the alkenyl groups are shown by the following structures: H₂C═CH—, H₂C═CHCH₂—, H₂C═C(CH₃)CH₂—, H₂C═CHCH₂CH₂—, H₂C═CHCH₂CH₂CH₂—, and H₂C═CHCH₂CH₂CH₂CH₂—. Representative examples of alkynyl groups are shown by the following structures: HC≡C—, HC≡CCH₂—, HC≡CC(CH₃)—, HC≡CC(CH₃)₂—, HC≡CC(CH₃)₂CH₂—.

Component (D¹) may be selected from alpha olefins such as 1-hexene, 1-octene, 1-decene, 1-undecene, 1-decadecene; branched allyl hydrocarbons such as 2-propyl-1-heptene; and similar homologs. Component (D¹) may also be selected from aryl containing hydrocarbons such as alphamethylstyrene.

Component (D²) may be selected from those polyoxyalkylenes having the average formula

R³O—[(C₂H₄O)_{c′}(C₃H₆O)_{d′}(C₄H₃O)ₑ]—R⁴

where R³ is a monovalent unsaturated aliphatic hydrocarbon group containing 2 to 12 carbon atoms, c′ is from 0 to 100, d′ is from 0 to 100, e is from 0 to 100, providing the sum of c′, d′, and e is >0. R⁴ is hydrogen, an acyl group, or a monovalent hydrocarbon group containing 1 to 8 carbons.

Representative, non-limiting examples of polyoxyalkylenes, useful as component (D²) include:

H₂C═CHCH₂O(C₂H₄O)_{c′}H

H₂C═CHCH₂O(C₂H₄O)_{c′}CH₃

H₂C═CHCH₂O(C₂H₄O)_{c′}C(O)CH₃

H₂C═CHCH₂O(C₂H₄O)_{c′}(C₃H₆O)_{d′}H

H₂C═CHCH₂O(C₂H₄O)_{c′}(C₃H₆O)_{d′}CH₃

H₂C═C(CH₃)CH₂O(C₂H₄O)_{c′}H

H₂C═CHC(CH₃)₂O(C₂H₄O)_{c′}H

H₂C═C(CH₃)CH₂O(C₂H₄O)_{c′}CH₃

H₂C═C(CH₃)CH₂O(C₂H₄O)_{c′}(C₃H₆O)_{d′}H

H₂C═C(CH₃)CH₂O(C₂H₄O)_{c′}(C₃H₆O)_{d′}CH₃

H₂C═C(CH₃)CH₂O(C₂H₄O)_{c′}C(O)CH₃

HC≡CCH₂O(C₂H₄O)_{c′}H

HC≡CCH₂O(C₂H₄O)_{c′}CH₃

HC≡CCH₂O(C₂H₄O)_{c′}(C₃H₆O)_{d′}H

HC≡CCH₂O(C₂H₄O)_{c′}(C₃H₆O)_{d′}CH₃

HC≡CCH₂O(C₂H₄O)_{c′}C(O)CH₃

where c′ and d′ are as defined above.

Component (D³) may be selected from XZ′ₙ derivatives which contain only one unsaturated aliphatic group, such as the reaction product of a secondary amine and an ethylenically-unsaturated epoxide containing only one unsaturated aliphatic group, for example dialkylamine may react with 1 allyl glycidyl ether, providing for a XZ′ derivative comprising 1 unsaturated aliphatic group. Component (D³) may also be the reaction product of a primary amine and an ethylenically-unsaturated epoxide containing only one unsaturated aliphatic group, where stoechiometry is controlled so that only one epoxide reacts with the amine, and so only one unsaturated group is present on the amine.

Component (D⁴) may be selected from linear or branched siloxane polymer comprising one unsaturated aliphatic group, such as monovinyl terminated polydimethylsiloxane (M^{Vi}DₓM), monovinyl functional polydimethylsiloxane (MD_{X}D^{(Vi)}_{y}M), vinyltris(trimethylsiloxysilane), where x≧0 and y=1.

Component (D⁵) may be selected from polyol component comprising one unsaturated aliphatic group. Such polyols include glycerol, sorbitol, xylitol. Examples of component (D⁵) include allyl xylitol, 3-allyloxy-1,2-propanediol, and diglycerol monoallyl ether.

Component (D) may be added to the silicone organic elastomer either during its formation, i.e. simultaneously reacting components (A), (B), (C) and (D), in a first reaction, for example reacting a partial quantity of SiH groups of component (A) with (C) and (D), followed by further reaction with (B); or subsequently added to a formed silicone organic elastomer having SiH content, for example, from unreacted SiH units present on the silicone organic elastomer.

The amount of component (D) used in the hydrosilylation reaction may vary, providing the molar quantity of the total aliphatic unsaturated groups present in the reaction from components (B) and (D) is such that the molar ratio of the SiH units of component (A) to the aliphatic unsaturated groups of components (B) and (D) ranges from 10/1 to 1/10.

### ii) The Carrier Fluid

A silicone organic elastomer gel composition comprises the silicone organic elastomer i) in a carrier fluid ii). Typically, the carrier fluid is the solvent used in carrying out the hydrosilylation reaction to form the silicone organic elastomer. Suitable carrier fluids include, but are not limited to, organic liquids (oils and solvents), liquid organopolysiloxanes and mixtures of these.

Liquid organopolysiloxanes include linear and cyclic organopolysiloxanes, volatile and non-volatile organopolysiloxanes. Liquid organopolysiloxanes suitable as carrier fluid generally have a viscosity at 25° C. in the range of 1 to 1,000 mm²/sec.

Examples of suitable organopolysiloxanes include hexamethylcyclotrisiloxane, octamethylcyclotetrasiloxane, decamethylcyclopentasiloxane, dodecamethylcyclohexasiloxane, octamethyltrisiloxane, decamethyltetrasiloxane, dodecamethylpentasiloxane, tetradecamethylhexasiloxane, hexadeamethylheptasiloxane, heptamethyl-3-{(trimethylsilyl)oxy)}trisiloxane, hexamethyl-3,3,bis{(trimethylsilyl)oxy}trisiloxane pentamethyl{(trimethylsilyl)oxy}cyclotrisiloxane, polydimethylsiloxanes, polydiethylsiloxanes, polymethylethylsiloxanes, polymethylphenylsiloxanes, polydiphenylsiloxanes, and any mixtures thereof.

Organic liquids include those considered as oils or solvents. Examples of organic liquids include aliphatic hydrocarbons, aromatic hydrocarbons, alcohols, aldehydes, ketones, amines, esters, ethers, glycols, glycol ethers, alkyl halides and aromatic halides. Hydrocarbons include, isododecane, isohexadecane, isoparaffins (Isopar L (C11-C13), Isopar H (C11-C12)), hydrogenated polydecene. Ethers and esters include, isodecyl neopentanoate, neopentylglycol heptanoate, glycol distearate, dicaprylyl carbonate, diethylhexyl carbonate, propylene glycol n butyl ether, ethyl-3 ethoxypropionate, propylene glycol methyl ether acetate, tridecyl neopentanoate, propylene glycol methylether acetate (PGMEA), propylene glycol methylether (PGME), octyldodecyl neopentanoate, diisobutyl adipate, diisopropyl adipate, propylene glycol dicaprylate/dicaprate, and octyl palmitate.

Additional organic liquids suitable as carrier fluid include fats, oils, fatty acids, and fatty alcohols.

The amount of i) silicone organic elastomer and ii) carrier fluid is such that the composition contains 2-95 weight percent, alternatively 5 to 95 weight percent, alternatively 10 to 90 weight percent of i) the silicone organic elastomer, and 5-98 weight percent, alternatively 95 to 5 weight percent, alternatively 90 to 10 weight percent of ii) the carrier fluid, providing the sum of components i) and ii), and any other ingredients or components present in the composition, adds up to 100 weight percent.

### Process for Preparing the Gel Composition

The silicone organic elastomers gel compositions may be prepared by

i) reacting:
(A) an organohydrogensiloxane comprising siloxy units of average formula

(R¹₃SiO_{0.5})ᵥ(R²₂SiO)ₓ(R²HSiO)_{y}
wherein R¹ is hydrogen or R²,
R² is a monovalent hydrocarbyl
v≧2, x≧0, y≧1,
(B) a XZ′ₙ derivative comprising at least 2 unsaturated aliphatic groups,
(C) in the presence of a hydrosilylation catalyst, and optionally
(D) wherein (D) is selected from
   (D¹) a hydrocarbon containing 6-30 carbons having one unsaturated aliphatic hydrocarbon group,
   (D²) a polyoxyalkylene having one unsaturated aliphatic group,
   (D³) an XZ′ₙ derivative containing one unsaturated aliphatic group,
   (D⁴) a linear or branched siloxane polymer comprising one unsaturated aliphatic group,
   (D⁵) a polyol component comprising one unsaturated aliphatic group, and mixtures of (D¹), (D²), (D³), (D⁴) and/or (D⁵)
      ii) in the presence of a carrier fluid.

Components (A), (B), (C), and optionally (D) and the carrier fluid ii), and the quantities used in the process are the same as described above. The order of addition of components (A), (B), (C), and optionally (D) in step I) is not critical. Typically, components (A), (B), and optionally (D) are combined with the carrier fluid with mixing, and the mixture heated to 70-90° C. Then, the catalyst (C) is added to cause the hydrosilylation reaction. Alternatively, components (A) and (D) are combined, mixed, and heated to 70-90° C., catalyst (C) added, and subsequently component (B) is added.

The process of the present disclosure may further include the step of mixing an organovinylsiloxane to the gel composition. Organovinylsiloxanes are organopolysiloxanes having at least one vinyl (Vi or CH₂═CH—) containing siloxy unit, that is at least one siloxy unit in the organopolysiloxane has the formula (R₂ViSiO_{0.5}), (RViSiO), or (ViSiO_{1.5}). The addition of an organovinylsiloxane may enhance the long term stability of the gel composition. Although not wishing to be bound by any theory, the present inventors believe the addition of the organovinylsiloxane may react with residual SiH that may remain on the silicone organic elastomer.

Further components may be added to the silicone organic elastomer in view of improving stability such as anti-oxidants (such as tocopherol) when polyether groups are present, or catalyst inactivators such as triphenylphosphine.

### E) Additional Component (E)

An additional component may be added to the silicone organic elastomer gel composition either during the making of the silicone organic elastomer (pre-load method), or added after the formation of the silicone organic elastomer (post load method). Such component may be selected from any personal or health care active, as disclosed here further.

The amount of component (E) present in the silicone organic elastomer gel composition may vary, but typically range as follows: 0.05 to 50 wt %, alternatively 1 to 25 wt %, or alternatively 1 to 10 wt %, based on the total amount by weight of silicone organic elastomer gel composition.

### Gel Paste Compositions Containing the Silicone Organic Elastomer

The silicone organic elastomer gel compositions of the present invention can be used to prepare gel paste compositions by:
I) shearing the silicone organic elastomer gel, as described above,
II) combining the sheared silicone organic elastomer gel with additional quantities of ii) the carrier fluid, as described above, and optionally the component (E) to form a gel paste composition.

The silicone organic elastomer gel compositions of the present invention may be considered as discrete crosslinked silicone organic elastomers dispersed in carrier fluids. The silicone organic elastomer gel compositions are also effective rheological thickeners for many organic and silicone fluids. As such they can be used to prepare useful gel blend compositions, such as “paste” compositions.

To make such silicone organic elastomer pastes, the aforementioned silicone organic elastomer gels of known initial elastomer content are sheared to obtain small particle size and may optionally be further diluted to a final elastomer content. “Shearing”, as used herein refers to any shear mixing process, such as obtained from homogenizing, sonolating, or any other mixing processes known in the art as shear mixing. The shear mixing of the silicone organic elastomer gel composition results in a composition having reduced particle size. The subsequent composition having reduced particle size is then further combined with additional quantities of ii) the carrier fluid. Typically, the amount of carrier fluid added to the gel to form the gel paste is sufficient to provide a gel paste composition containing 30 wt % of the silicone organic elastomer, alternatively 20 wt %, or alternatively 10 wt %. The carrier fluid may be any carrier fluid as described above. The carrier fluid may be an organic liquid, such as those described above. The carrier fluid may be an organopolysiloxane having a viscosity at 25° C. in the range of 1 to 1,000 mm²/sec.

The technique for combining the ii) the carrier fluid with the silicone organic elastomer composition, and optionally component E), typically involves simple stirring or mixing. The resulting compositions may be considered as a paste, having a viscosity, at least 50 Pa·s, alternatively at least 250 Pa·s, or alternatively at least 400 Pa·s, at least 600 Pa·s, at least 1000 Pa·s, as measured on a Brookfield DVII+viscometer with Helipath attachment using spindle T-D (20.4 mm crossbar) at 2.5 rpm.

The silicone organic elastomer comprising an amino functional group, the gel and/or paste obtained therewith, is present in a cosmetic composition in conjunction with a cosmetic ingredient, optionally in a cosmetically acceptable medium.

Cosmetic compositions include those compositions which are intended to be placed in contact with the external parts of the human body (skin (epidermis), hair system, nails, mucosa, etc., also referred to as “keratinous substrates”) or with the teeth and the mucous membranes of the oral cavity with a view exclusively or mainly to cleaning them, perfuming them, changing their appearance, protecting them, keeping them in good condition or correcting body odours. In some instances, cosmetic compositions may also include health care compositions.

Cosmetic applications, and in some instances health care applications, include skin care, hair care, or nail care applications.

Cosmetic ingredients are those ingredients known to be used in cosmetic application. A wide review of such ingredients may be found in the CTFA cosmetic ingredient handbook.

Cosmetically acceptable medium include water, solvents, diluents, or mixtures and emulsions thereof.

Cosmetic ingredients include emollients, waxes, film formers, moisturizers, surface active materials such as surfactants or detergents or emulsifiers, thickeners, water phase stabilizing agents, pH controlling agents, preservatives and cosmetic biocides, sebum absorbants or sebum control agents, vegetable or botanical extracts, vitamins, proteins or amino-acids and their derivatives, pigments, colorants, fillers, silicone conditioning agents, cationic conditioning agents, hydrophobic conditioning agents, UV absorbers, sunscreen agents, antidandruff agents, antiperspirant agents, deodorant agents, skin protectants, hair dyes, nail care ingredients, fragrances or perfume, antioxidants, oxidizing agents, reducing agents, propellant gases, and mixtures thereof.

Additional ingredients that may be used in the cosmetic compositions include fatty alcohols, colour care additives, anticellulites, pearlising agents, chelating agents, styling agents, ceramides, suspending agents and others.

Health care ingredients include antiacne agents, antibacterial agents, antifungal agents, therapeutic active agents, external analgesics, skin bleaching agents, anti-cancer agents, diuretics, agents for treating gastric and duodenal ulcers, proteolytic enzymes, antihistamine or H1 histamine blockers, sedatives, bronchodilators, diluents.

Additional ingredients that may be used in the health care compositions include antibiotic, antiseptic, antibacterial, anti-inflammatory, astringents, hormones, smoking cessation compositions, cardiovascular, antiarrythmic, alpha-I blocker, beta blocker, ACE inhibitor, antiaggregant, non-steroidal anti-inflammatory agents such as diclofenac, antipsoriasis agents such as clobetasol propionate, antidermatitis agents, tranquillizer, anticonvulsant, anticoagulant agents, healing factors, cell growth nutrients, peptides, corticosteroidal drugs, antipruritic agents and others.

Cosmetic ingredients may be used in health care compositions, such as waxes, and others; and health care ingredients may be used in cosmetic compositions such as anti-acne agents, and others.

Examples of emollients include volatile or non-volatile silicone oils and gums; dimethicone, dimethiconol; silicone resins such as polypropylsilsesquioxane and phenyl trimethicone; silicone elastomers such as dimethicone crosspolymer, PEG-12 dimethicone/PPG crosspolymer, dimethicone/bis-isobutyl PPG-20 crosspolymer; alkylmethylsiloxanes such as caprylyl methicone; volatile or non-volatile hydrocarbon compounds, such as squalene, paraffin oils, petrolatum oils and naphthalene oils; hydrogenated or partially hydrogenated polyisobutene; isoeicosane; squalane; isoparaffin; isododecane; isodecane or isohexa-decane; branched C8-C16 esters; isohexyl neopentanoate; ester oils such as isononyl isononanoate, cetostearyl octanoate, isopropyl myristate, palmitate derivatives, stearates derivatives, isostearyl isostearate and the heptanoates, octanoates, decanoates or ricinoleates of alcohols or of polyalcohols, or mixtures thereof; hydrocarbon oils of plant origin, such as wheatgerm, sunflower, grapeseed, castor, shea, avocado, olive, soybean, sweet almond, palm, rapeseed, cotton seed, hazelnut, macadamia, jojoba, blackcurrant, evening primrose; or triglycerides of caprylic/capric acids; higher fatty acids, such as oleic acid, linoleic acid or linolenic acid, and mixtures thereof.

Example of waxes include hydrocarbon waxes such as beeswax, lanolin wax, rice wax, carnauba wax, candelilla wax, microcrystalline waxes, paraffins, ozokerite, polyethylene waxes, synthetic wax, ceresin, lanolin, lanolin derivatives, cocoa butter, shellac wax, bran wax, capok wax, sugar cane wax, montan wax, whale wax, bayberry wax, silicone waxes (e.g. polymethylsiloxane alkyls, alkoxys and/or esters, C30-45 alkyldimethylsilyl polypropylsilsesquioxane, C30-45 alkyl methicone), and mixtures thereof.

A film former may be understood as a polymer capable, by itself or in presence of an auxiliary film-forming agent, of forming a macroscopically continuous film on a support (especially keratinous material), typically a cohesive film or alternatively, a film whose cohesion and mechanical properties are such that the said film can be isolated from said support. Film formers include silicone resins such as polypropylsilsesquioxane, trimethylsiloxysilicate; silicone acrylate copolymers such as acrylates/polytrimethylsiloxymethacrylate copolymer.

Examples of moisturizers include lower molecular weight aliphatic diols such as propylene glycol and butylene glycol; polyols such as glycerine and sorbitol; and polyoxyethylene polymers such as polyethylene glycol 200; hyaluronic acid and its derivatives, and mixtures thereof.

Examples of surface active materials may be anionic, cationic or non ionic, and include organomodified silicones such as dimethicone copolyol alkyl dimethicone copolyol, alkyl Ttris(trimethylsiloxy)silylethyl dimethicone copolyol, bis polyether dimethicone copolymer, alkyl glyceryl tris(trimethylsiloxy)silylethyl dimethicone; oxyethylenated and/or oxypropylenated ethers of glycerol; oxyethylenated and/or oxypropylenated ethers of fatty alcohols such as ceteareth-30, C12-15 pareth-7; fatty acid esters of polyethylene glycol such as PEG-50 stearate, PEG-40 monostearate; saccharide esters and ethers, such as sucrose stearate, sucrose cocoate and sorbitan stearate, and mixtures thereof; phosphoric esters and salts thereof, such as DEA oleth-10 phosphate; sulphosuccinates such as disodium PEG-5 citrate lauryl sulphosuccinate and disodium ricinoleamido MEA sulphosuccinate; alkyl ether sulphates, such as sodium lauryl ether sulphate; isethionates; betaine derivatives; and mixtures thereof.

Further examples of nonionic surfactants include polyoxyethylene alkyl ethers, polyoxyethylene alkylphenol ethers, polyoxyethylene lauryl ethers, polyoxyethylene sorbitan monoleates, polyoxyethylene alkyl esters, polyoxyethylene sorbitan alkyl esters, polyethylene glycol, polypropylene glycol, diethylene glycol, ethoxylated trimethylnonanols, polyoxyalkylene-substituted silicones (rake or ABn types), silicone alkanolamides, silicone esters, silicone glycosides, silicone functionalized saccharides and mixtures thereof.

Nonionic surfactants include dimethicone copolyols, fatty acid esters of polyols, for instance sorbitol or glyceryl mono-, di-, tri- or sesquioleates or stearates, glyceryl or polyethylene glycol laurates; fatty acid esters of polyethylene glycol (polyethylene glycol monostearate or monolaurate); polyoxyethylenated fatty acid esters (stearate or oleate) of sorbitol; polyoxyethylenated alkyl (lauryl, cetyl, stearyl or octyl)ethers.

Anionic surfactants include carboxylates (sodium 2-(2-hydroxyalkyloxy)acetate)), amino acid derivatives (N-acylglutamates, N-acylgly-cinates or acylsarcosinates), alkyl sulfates, alkyl ether sulfates and oxyethylenated derivatives thereof, sulfonates, isethionates and N-acylisethionates, taurates and N-acyl N-methyltaurates, sulfosuccinates, alkylsulfoacetates, phosphates and alkyl phosphates, polypeptides, anionic derivatives of alkyl polyglycoside (acyl-D-galactoside uronate), and fatty acid soaps, and mixtures thereof.

Amphoteric and zwitterionic surfactants include betaines, N-alkylamidobetaines and derivatives thereof, proteins and derivatives thereof, glycine derivatives, sultaines, alkyl polyaminocarboxylates and alkylamphoacetates, and mixtures thereof.

Examples of thickeners include acrylamide copolymers, acrylate copolymers and salts thereof (such as sodium polyacrylate), xanthan gum and derivatives, cellulose gum and cellulose derivatives (such as methylcellulose, methylhydroxypropylcellulose, hydroxypropylcellulose, polypropylhydroxyethylcellulose), starch and starch derivatives (such as hydroxyethylamylose and starch amylase), polyoxyethylene, carbomer, sodium alginate, arabic gum, cassia gum, guar gum and guar gum derivatives, cocamide derivatives, alkyl alcohols, gelatin, PEG-derivatives, saccharides (such as fructose, glucose) and saccharides derivatives (such as PEG-120 methyl glucose diolate), and mixtures thereof.

Examples of water phase stabilizing agents include electrolytes (e.g. alkali metal salts and alkaline earth salts, especially the chloride, borate, citrate, and sulfate salts of sodium, potassium, calcium and magnesium, as well as aluminum chlorohydrate, and polyelectrolytes, especially hyaluronic acid and sodium hyaluronate), polyols (glycerine, propylene glycol, butylene glycol, and sorbitol), alcohols such as ethyl alcohol, and hydrocolloids, and mixtures thereof.

Examples of pH controlling agents include any water soluble acid such as a carboxylic acid or a mineral acid such as hydrochloric acid, sulphuric acid, and phosphoric acid, monocarboxylic acid such as acetic acid and lactic acid, and polycarboxylic acids such as succinic acid, adipic acid, citric acid, and mixtures thereof.

Example of preservatives and cosmetic biocides include paraben derivatives, hydantoin derivatives, chlorhexidine and its derivatives, imidazolidinyl urea, phenoxyethanol, silver derivatives, salicylate derivatives, triclosan, ciclopirox olamine, hexamidine, oxyquinoline and its derivatives, PVP-iodine, zinc salts and derivatives such as zinc pyrithione, and mixtures thereof.

Examples of sebum absorbants or sebum control agents include silica silylate, silica dimethyl silylate, dimethicone/vinyl dimethicone crosspolymer, polymethyl methacrylate, cross-linked methylmethacrylate, aluminum starch octenylsuccinate, and mixtures thereof.

Examples of vegetable or botanical extracts are derived from plants (herbs, roots, flowers, fruits, or seeds) in oil or water soluble form, such as coconut, green tea, white tea, black tea, horsetail, ginkgo biloba, sunflower, wheat germ, seaweed, olive, grape, pomegranate, aloe, apricot kernel, apricot, carrot, tomato, tobacco, bean, potato, actzuki bean, catechu, orange, cucumber, avocado, watermelon, banana, lemon or palm. Examples of herbal extracts include dill, horseradish, oats, neem, beet, broccoli, tea, pumpkin, soybean, barley, walnut, flax, ginseng, poppy, avocado, pea, sesame, and mixtures thereof.

Examples of vitamins include a variety of different organic compounds such as alcohols, acids, sterols, and quinones. They may be classified into two solubility groups: lipid-soluble vitamins and water-soluble vitamins. Lipid-soluble vitamins that have utility in personal care formulations include retinol (vitamin A), ergocalciferol (vitamin D2), cholecalciferol (vitamin D3), phytonadione (vitamin K1), and tocopherol (vitamin E). Water-soluble vitamins that have utility in personal care formulations include ascorbic acid (vitamin C), thiamin (vitamin B1) niacin (nicotinic acid), niacinamide (vitamin B3), riboflavin (vitamin B2), pantothenic acid (vitamin B5), biotin, folic acid, pyridoxine (vitamin B6), and cyanocobalamin (vitamin B12). Additional examples of vitamins include derivatives of vitamins such as retinyl palmitate (vitamin A palmitate), retinyl acetate (vitamin A acetate), retinyl linoleate (vitamin A linoleate), and retinyl propionate (vitamin A propionate), tocopheryl acetate (vitamin E acetate), tocopheryl linoleate (vitamin E linoleate), tocopheryl succinate (vitamin E succinate), tocophereth-5, tocophereth-10, tocophereth-12, tocophereth-18, tocophereth-50 (ethoxylated vitamin E derivatives), PPG-2 tocophereth-5, PPG-5 tocophereth-2, PPG-10 tocophereth-30, PPG-20 tocophereth-50, PPG-30 tocophereth-70, PPG-70 tocophereth-100 (propoxylated and ethoxylated vitamin E derivatives), sodium tocopheryl phosphate, ascorbyl palmitate, ascorbyl dipalmitate, ascorbyl glucoside, ascorbyl tetraisopalmitate, tetrahexadecyl ascorbate, ascorbyl tocopheryl maleate, potassium ascorbyl tocopheryl phosphate, tocopheryl nicotinate, and mixtures thereof.

Examples of proteins or amino-acids and their derivatives include those extracted from wheat, soy, rice, corn, keratin, elastin or silk. Proteins may be in the hydrolyzed form and they may also be quaternized, such as hydrolyzed elastin, hydrolyzed wheat powder, hydrolyzed silk. Examples of protein include enzymes such as hydrolases, cutinases, oxidases, transferases, reductases, hemicellulases, esterases, isomerases, pectinases, lactases, peroxidases, laccases, catalases, and mixtures thereof. Examples of hydrolases include proteases (bacterial, fungal, acid, neutral or alkaline), amylases (alpha or beta), lipases, mannanases, cellulases, collagenases, lisozymes, superoxide dismutase, catalase, and mixtures thereof.

Examples of pigments and colorants include surface treated or untreated iron oxides, surface treated or untreated titanium dioxide, surface treated or untreated mica, silver oxide, silicates, chromium oxides, carotenoids, carbon black, ultramarines, chlorophyllin derivatives and yellow ocher. Examples of organic pigments include aromatic types including azo, indigoid, triphenylmethane, anthraquinone, and xanthine dyes which are designated as D&C and FD&C blues, browns, greens, oranges, reds, yellows, etc, and mixtures thereof. Surface treatments of pigments include those based on silicone, fluorine.

Examples of fillers include talc, micas, kaolin, zinc or titanium oxides, calcium or magnesium carbonates, silica, silica silylate, titanium dioxide, glass or ceramic beads, polymethylmethacrylate beads, boron nitride, aluminum silicate, aluminum starch octenylsuccinate, bentonite, magnesium aluminum silicate, nylon, silk powder metal soaps derived from carboxylic acids having 8-22 carbon atoms, non-expanded synthetic polymer powders, expanded powders and powders from natural organic compounds, such as cereal starches, which may or may not be crosslinked, copolymer microspheres, polytrap, silicone resin microbeads, and mixtures thereof. The fillers may be surface treated to modify affinity or compatibility with remaining ingredients.

Examples of silicone conditioning agents include silicone oils such as dimethicone; silicone gums such as dimethiconol; silicone resins such as trimethylsiloxy silicate, polypropyl silsesquioxane; silicone elastomers; alkylmethylsiloxanes; organomodified silicone oils, such as amodimethicone, aminopropyl phenyl trimethicone, phenyl trimethicone, trimethyl pentaphenyl trisiloxane, silicone quaternium-16/glycidoxy dimethicone crosspolymer, silicone quaternium-16; saccharide functional siloxanes; carbinol functional siloxanes; silicone polyethers; siloxane copolymers (divinyldimethicone/dimethicone copolymer); acrylate or acrylic functional siloxanes; and mixtures or emulsions thereof.

Examples of cationic conditioning agents include guar derivatives such as hydroxypropyltrimethylammonium derivative of guar gum; cationic cellulose derivatives, cationic starch derivatives; quaternary nitrogen derivatives of cellulose ethers;

homopolymers of dimethyldiallyl ammonium chloride; copolymers of acrylamide and dimethyldiallyl ammonium chloride; homopolymers or copolymers derived from acrylic acid or methacrylic acid which contain cationic nitrogen functional groups attached to the polymer by ester or amide linkages; polymeric quaternary ammonium salts of hydroxyethyl cellulose reacted with a fatty alkyl dimethyl ammonium substituted epoxide; polycondensation products of N,N′-bis-(2,3-epoxypropyl)-piperazine or piperazine-bis-acrylamide and piperazine; and copolymers of vinylpyrrolidone and acrylic acid esters with quaternary nitrogen functionality. Specific materials include the various polyquats Polyquaternium-7, Polyquaternium-8, Polyquaternium-10, Polyquaternium-11, and Polyquaternium-23. Other categories of conditioners include cationic surfactants such as cetyl trimethylammonium chloride, cetyl trimethylammonium bromide, stearyltrimethylammonium chloride, and mixtures thereof. In some instances, the cationic conditioning agent is also hydrophobically modified, such as hydrophobically modified quaternized hydroxyethylcellulose polymers; cationic hydrophobically modified galactomannan ether; and mixtures thereof.

Examples of hydrophobic conditioning agents include guar derivatives; galactomannan gum derivatives; cellulose derivatives; and mixtures thereof.

UV absorbers and sunscreen agents include those which absorb ultraviolet light between about 290-320 nanometers (the UV-B region) and those which absorb ultraviolet light in the range of 320-400 nanometers (the UV-A region).

Some examples of sunscreen agents are aminobenzoic acid, cinoxate, diethanolamine methoxycinnamate, digalloyl trioleate, dioxybenzone, ethyl 4-[bis(Hydroxypropyl)]aminobenzoate, glyceryl aminobenzoate, homosalate, lawsone with dihydroxyacetone, menthyl anthranilate, octocrylene, ethyl hexyl methoxycinnamate, octyl salicylate, oxybenzone, padimate O, phenylbenzimidazole sulfonic acid, red petrolatum, sulisobenzone, titanium dioxide, trolamine salicylate, and mixtures thereof.

Some examples of UV absorbers are acetaminosalol, allatoin PABA, benzalphthalide, benzophenone, benzophenone 1-12, 3-benzylidene camphor, benzylidenecamphor hydrolyzed collagen sulfonamide, benzylidene camphor sulfonic Acid, benzyl salicylate, bornelone, bumetriozole, butyl Methoxydibenzoylmethane, butyl PABA, ceria/silica, ceria/silica talc, cinoxate, DEA-methoxycinnamate, dibenzoxazol naphthalene, di-t-butyl hydroxybenzylidene camphor, digalloyl trioleate, diisopropyl methyl cinnamate, dimethyl PABA ethyl cetearyldimonium tosylate, dioctyl butamido triazone, diphenyl carbomethoxy acetoxy naphthopyran, disodium bisethylphenyl tiamminotriazine stilbenedisulfonate, disodium distyrylbiphenyl triaminotriazine stilbenedisulfonate, disodium distyrylbiphenyl disulfonate, drometrizole, drometrizole trisiloxane, ethyl dihydroxypropyl PABA, ethyl diisopropylcinnamate, ethyl methoxycinnamate, ethyl PABA, ethyl urocanate, etrocrylene ferulic acid, glyceryl octanoate dimethoxycinnamate, glyceryl PABA, glycol salicylate, homosalate, isoamyl p-methoxycinnamate, isopropylbenzyl salicylate, isopropyl dibenzolylmethane, isopropyl methoxycinnamate, menthyl anthranilate, menthyl salicylate, 4-methylbenzylidene, camphor, octocrylene, octrizole, octyl dimethyl PABA, ethyl hexyl methoxycinnamate, octyl salicylate, octyl triazone, PABA, PEG-25 PABA, pentyl dimethyl PABA, phenylbenzimidazole sulfonic acid, polyacrylamidomethyl benzylidene camphor, potassium methoxycinnamate, potassium phenylbenzimidazole sulfonate, red petrolatum, sodium phenylbenzimidazole sulfonate, sodium urocanate, TEA-phenylbenzimidazole sulfonate, TEA-salicylate, terephthalylidene dicamphor sulfonic acid, titanium dioxide, tri PABA panthenol, urocanic acid, VA/crotonates/methacryloxybenzophenone-1 copolymer, and mixtures thereof.

Examples of antidandruff agents include pyridinethione salts, selenium compounds such as selenium disulfide, and soluble antidandruff agents, and mixtures thereof.

Examples of antiperspirant agents and deodorant agents include aluminum chloride, aluminum zirconium tetrachlorohydrex GLY, aluminum zirconium tetrachlorohydrex PEG, aluminum chlorohydrex, aluminum zirconium tetrachlorohydrex PG, aluminum chlorohydrex PEG, aluminum zirconium trichlorohydrate, aluminum chlorohydrex PG, aluminum zirconium trichlorohydrex GLY, hexachlorophene, benzalkonium chloride, aluminum sesquichlorohydrate, sodium bicarbonate, aluminum sesquichlorohydrex PEG, chlorophyllin-copper complex, triclosan, aluminum zirconium octachlorohydrate, zinc ricinoleate, and mixtures thereof.

Examples of skin protectants include allantoin, aluminium acetate, aluminium hydroxide, aluminium sulfate, calamine, cocoa butter, cod liver oil, colloidal oatmeal, dimethicone, glycerin, kaolin, lanolin, mineral oil, petrolatum, shark liver oil, sodium bicarbonate, talc, witch hazel, zinc acetate, zinc carbonate, zinc oxide, and mixtures thereof.

Examples of hair dyes include 1-acetoxy-2-methylnaphthalene; acid dyes; 5-amino-4-chloro-o-cresol; 5-amino-2,6-dimethoxy-3-hydroxypyridine; 3-amino-2,6-dimethylphenol; 2-amino-5-ethylphenol HCl; 5-amino-4-fluoro-2-methylphenol sulfate; 2-amino-4-hydroxyethylaminoanisole; 2-amino-4-hydroxyethylaminoanisole sulfate; 2-amino-5-nitrophenol; 4-amino-2-nitrophenol; 4-amino-3-nitrophenol; 2-amino-4-nitrophenol sulfate; m-aminophenol HCl; p-aminophenol HCl; m-aminophenol; o-aminophenol; 4,6-bis(2-hydroxyethoxy)-m-phenylenediamine HCl; 2,6-bis(2-hydroxyethoxy)-3,5-pyridinediamine HCl; 2-chloro-6-ethylamino-4-nitrophenol; 2-chloro-5-nitro-N-hydroxyethyl p-phenylenediamine; 2-chloro-p-phenylenediamine; 3,4-diaminobenzoic acid; 4,5-diamino-1-((4-chlorophenyl)methyl)-1H-pyrazole-sulfate; 2,3-diaminodihydropyrazolo pyrazolone dimethosulfonate; 2,6-diaminopyridine; 2,6-diamino-3-((pyridin-3-yl)azo)pyridine; dihydroxyindole; dihydroxyindoline; N,N-dimethyl-p-phenylenediamine; 2,6-dimethyl-p-phenylenediamine; N,N-dimethyl-p-phenylenediamine sulfate; direct dyes; 4-ethoxy-m-phenylenediamine sulfate; 3-ethylamino-p-cresol sulfate; N-ethyl-3-nitro PABA; gluconamidopropyl aminopropyl dimethicone; Haematoxylon brasiletto wood extract; HC dyes; Lawsonia inermis (Henna) extract; hydroxyethyl-3,4-methylenedioxyaniline HCl; hydroxyethyl-2-nitro-p-toluidine; hydroxyethyl-p-phenylenediamine sulfate; 2-hydroxyethyl picramic acid; hydroxypyridinone; hydroxysuccinimidyl C21-22 isoalkyl acidate; isatin; Isatis tinctoria leaf powder; 2-methoxymethyl-p-phenylenediamine sulfate; 2-methoxy-p-phenylenediamine sulfate; 6-methoxy-2,3-pyridinediamine HCl; 4-methylbenzyl 4,5-diamino pyrazole sulfate; 2,2′-methylenebis 4-aminophenol; 2,2′-methylenebis-4-aminophenol HCl; 3,4-methylenedioxyaniline; 2-methylresorcinol; methylrosanilinium chloride; 1,5-naphthalenediol; 1,7-naphthalenediol; 3-nitro-p-Cresol; 2-nitro-5-glyceryl methylaniline; 4-nitroguaiacol; 3-nitro-p-hydroxyethylaminophenol; 2-nitro-N-hydroxyethyl-p-anisidine; nitrophenol; 4-nitrophenyl aminoethylurea; 4-nitro-o-phenylenediamine dihydrochloride; 2-nitro-p-phenylenediamine dihydrochloride; 4-nitro-o-phenylenediamine HCl; 4-nitro-m-phenylenediamine; 4-nitro-o-phenylenediamine; 2-nitro-p-phenylenediamine; 4-nitro-m-phenylenediamine sulfate; 4-nitro-o-phenylenediamine sulfate; 2-nitro-p-phenylenediamine sulfate; 6-nitro-2,5-pyridinediamine; 6-nitro-o-toluidine; PEG-3 2,2′-di-p-phenylenediamine; p-phenylenediamine HCl; p-phenylenediamine sulfate; phenyl methyl pyrazolone; N-phenyl-p-phenylenediamine HCl; pigment blue 15:1; pigment violet 23; pigment yellow 13; pyrocatechol; pyrogallol; resorcinol; sodium picramate; sodium sulfanilate; solvent yellow 85; solvent yellow 172; tetraaminopyrimidine sulfate; tetrabromophenol blue; 2,5,6-triamino-4-pyrimidinol sulfate; 1,2,4-trihydroxybenzene.

Example of nail care ingredients include butyl acetate; ethyl acetate; nitrocellulose; acetyl tributyl citrate; isopropyl alcohol; adipic acid/neopentyl glycol/trimelitic anhydride copolymer; stearalkonium bentonite; acrylates copolymer; calcium pantothenate; Cetraria islandica extract; Chondrus crispus; styrene/acrylates copolymer; trimethylpentanediyl dibenzoate-1; polyvinyl butyral; N-butyl alcohol; propylene glycol; butylene glycol; mica; silica; tin oxide; calcium borosilicate; synthetic fluorphlogopite; polyethylene terephtalate; sorbitan laurate derivatives; talc; jojoba extract; diamond powder; isobutylphenoxy epoxy resin; silk powder; and mixtures thereof.

Examples of fragrances or perfume include hexyl cinnamic aldehyde; anisaldehyde; methyl-2-n-hexyl-3-oxo-cyclopentane carboxylate; dodecalactone gamma; methylphenylcarbinyl acetate; 4-acetyl-6-tert-butyl-1,1-dimethyl indane; patchouli; olibanum resinoid; labdanum; vetivert; copaiba balsam; fir balsam; 4-(4-hydroxy-4-methyl pentyl)-3-cyclohexene-1-carboxaldehyde; methyl anthranilate; geraniol; geranyl acetate; linalool; citronellol; terpinyl acetate; benzyl salicylate; 2-methyl-3-(p-isopropylphenyl)-propanal; phenoxyethyl isobutyrate; cedryl acetal; aubepine; musk fragrances; macrocyclic ketones; macrolactone musk fragrances; ethylene brassylate; and mixtures thereof. Further perfume ingredients are described in detail in standard textbook references such as Perfume and Flavour Chemicals, 1969, S. Arctander, Montclair, N.J.

Examples of antioxidants are acetyl cysteine, arbutin, ascorbic acid, ascorbic acid polypeptide, ascorbyl dipalmitate, ascorbyl methylsilanol pectinate, ascorbyl palmitate, ascorbyl stearate, BHA, p-hydroxyanisole, BHT, t-butyl hydroquinone, caffeic acid, Camellia sinensis Oil, chitosan ascorbate, chitosan glycolate, chitosan salicylate, chlorogenic acids, cysteine, cysteine HCl, decyl mercaptomethylimidazole, erythorbic acid, diamylhydroquinone, di-t-butylhydroquinone, dicetyl thiodipropionate, dicyclopentadiene/t-butylcresol copolymer, digalloyl trioleate, dilauryl thiodipropionate, dimyristyl thiodipropionate, dioleyl tocopheryl methylsilanol, isoquercitrin, diosmine, disodium ascorbyl sulfate, disodium rutinyl disulfate, distearyl thiodipropionate, ditridecyl thiodipropionate, dodecyl gallate, ethyl ferulate, ferulic acid, hydroquinone, hydroxylamine HCl, hydroxylamine sulfate, isooctyl thioglycolate, kojic acid, madecassicoside, magnesium ascorbate, magnesium ascorbyl phosphate, melatonin, methoxy-PEG-7 rutinyl succinate, methylene di-t-butylcresol, methylsilanol ascorbate, nordihydroguaiaretic acid, octyl gallate, phenylthioglycolic acid, phloroglucinol, potassium ascorbyl tocopheryl phosphate, thiodiglycolamide, potassium sulfite, propyl gallate, rosmarinic acid, rutin, sodium ascorbate, sodium ascorbyl/cholesteryl phosphate, sodium bisulfite, sodium erythorbate, sodium metabisulfide, sodium sulfite, sodium thioglycolate, sorbityl furfural, tea tree (Melaleuca aftemifolia) oil, tocopheryl acetate, tetrahexyldecyl ascorbate, tetrahydrodiferuloylmethane, tocopheryl linoleate/oleate, thiodiglycol, tocopheryl succinate, thiodiglycolic acid, thioglycolic acid, thiolactic acid, thiosalicylic acid, thiotaurine, retinol, tocophereth-5, tocophereth-10, tocophereth-12, tocophereth-18, tocophereth-50, tocopherol, tocophersolan, tocopheryl linoleate, tocopheryl nicotinate, tocoquinone, o-tolyl biguanide, tris(nonylphenyl) phosphite, ubiquinone, zinc dibutyldithiocarbamate, and mixtures thereof.

Examples of oxidizing agents are ammonium persulfate, calcium peroxide, hydrogen peroxide, magnesium peroxide, melamine peroxide, potassium bromate, potassium caroate, potassium chlorate, potassium persulfate, sodium bromate, sodium carbonate peroxide, sodium chlorate, sodium iodate, sodium perborate, sodium persulfate, strontium dioxide, strontium peroxide, urea peroxide, zinc peroxide, and mixtures thereof.

Examples of reducing agents are ammonium bisufite, ammonium sulfite, ammonium thioglycolate, ammonium thiolactate, cystemaine HCl, cystein, cysteine HCl, ethanolamine thioglycolate, glutathione, glyceryl thioglycolate, glyceryl thioproprionate, hydroquinone, p-hydroxyanisole, isooctyl thioglycolate, magnesium thioglycolate, mercaptopropionic acid, potassium metabisulfite, potassium sulfite, potassium thioglycolate, sodium bisulfite, sodium hydrosulfite, sodium hydroxymethane sulfonate, sodium metabisulfite, sodium sulfite, sodium thioglycolate, strontium thioglycolate, superoxide dismutase, thioglycerin, thioglycolic acid, thiolactic acid, thiosalicylic acid, zinc formaldehyde sulfoxylate, and mixtures thereof.

Examples of propellant gases include carbon dioxide, nitrogen, nitrous oxide, volatile hydrocarbons such as butane, isobutane, or propane, and chlorinated or fluorinated hydrocarbons such as dichlorodifluoromethane and dichlorotetrafluoroethane or dimethylether; and mixtures thereof.

Examples of antiacne agents include salicylic acid, sulfur benzoyl, peroxide, tretinoin, and mixtures thereof.

Examples of antibacterial agents include chlorohexadiene gluconate, alcohol, benzalkonium chloride, benzethonium chloride, hydrogen peroxide, methylbenzethonium chloride, phenol, poloxamer 188, povidone-iodine, and mixtures thereof.

Examples of antifungal agents include miconazole nitrate, calcium undecylenate, undecylenic acid, zinc undecylenate, and mixtures thereof.

Examples of therapeutic active agents include penicillins, cephalosporins, tetracyclines, macrolides, epinephrine, amphetamines, aspirin, acetominophen, barbiturates, catecholamines, benzodiazepine, thiopental, codeine, morphine, procaine, lidocaine, benzocaine, sulphonamides, ticonazole, perbuterol, furosamide, prazosin, hormones, prostaglandins, carbenicillin, salbutamol, haloperidol, suramin, indomethicane, diclofenac, glafenine, dipyridamole, theophylline, hydrocortisone, steroids, scopolamine, and mixtures thereof.

Examples of external analgesics are benzyl alcohol, capsicum oleoresin (Capsicum frutescens oleoresin), methyl salicylate, camphor, phenol, capsaicin, juniper tar (Juniperus oxycedrus tar), phenolate sodium (sodium phenoxide), capsicum (Capsicum frutescens), menthol, resorcinol, methyl nicotinate, turpentine oil (turpentine), and mixtures thereof.

An example of a skin bleaching agent is hydroquinone.

Examples of anti-cancer agents include alkylating agents (such as busulfan, fluorodopan), antimitotic agents (such as colchicine, rhizoxin), topoisomerase I inhibitors (such as camptothecin and its derivatives), topoisomerase II inhibitors (such as menogaril, amonafide), RNA/DNA or DNA anti-metabolites (such as acivicin, guuanazole), plant alkaloids and terpenoids, antineoplastics, some plant-derived compounds (such as podophyllotoxin, vinca alkaloids), and mixtures thereof.

Examples of diuretics include loop diuretics (such as bumetanide, furosemide), thiazide diuretics (such as chlorothiazide, hydroflumethiazide), potassium-sparing diuretics (such as amioloride, spironolactone), carbonic anhydrase inhibitors (such as acetazolamide), osmotic diuretics (such as mannitol), and mixtures thereof.

Examples of agents for treating gastric and duodenal ulcers include proton pump inhibitor (such as lansoprazole, omeprazole), acid blockers or H2 histamine blockers (such as cimetidine, ranitidine), bismuth, sucralfate, and mixtures thereof.

Examples of proteolytic enzymes include nattokinase, serratiopeptidase, bromelain, papain, and mixtures thereof.

Examples of antihistamine or H1 histamine blockers include brompheniramine, clemastine, cetirizine, loratadine, fexofenadine, and mixtures thereof.

Examples of sedatives include barbiturates (such as phenobarbitol), benzodiazepines (such as lorazepam), herbal sedatives, benzodiazepine-like drugs (such as zolpidem, zopiclone), and mixtures thereof.

Examples of bronchodilators include short-acting β2-agonists and long-acting β2-agonists, anticholinergics, and mixtures thereof.

The formulations of the present invention also include diluents. Such diluents are often necessary to decrease the viscosity of the formulation sufficiently for application.

Examples of diluents include silicon containing diluents such as hexamethyldisiloxane, octamethyltrisiloxane, and other short chain linear siloxanes such as octamethyltrisiloxane, decamethyltetrasiloxane, dodecamethylpentasiloxane, tetradecamethylhexasiloxane, hexadeamethylheptasiloxane, heptamethyl-3-{(trimethylsilyl)oxy)}trisiloxane, cyclic siloxanes such as hexamethylcyclotrisiloxane, octamethylcyclotetrasiloxane, decamethylcyclopentasiloxane, dodecamethylcyclohexasiloxane; volatile alkylmethylsiloxane such as caprylyl methicone; organic diluents such as butyl acetate, alkanes, alcohols, ketones, esters, ethers, glycols, glycol ethers, hydrofluorocarbons or any other material which can dilute the formulation without adversely affecting any of the component materials of the cosmetic composition. Hydrocarbons include isododecane, isohexadecane, Isopar L (C11-C13), Isopar H (C11-C12), hydrogenated polydecene. Ethers and esters include isodecyl neopentanoate, neopentylglycol heptanoate, glycol distearate, dicaprylyl carbonate, diethylhexyl carbonate, propylene glycol n butyl ether, ethyl-3 ethoxypropionate, propylene glycol methyl ether acetate, tridecyl neopentanoate, propylene glycol methylether acetate (PGMEA), propylene glycol methylether (PGME), octyldodecyl neopentanoate, diisobutyl adipate, diisopropyl adipate, propylene glycol dicaprylate/dicaprate, and octyl palmitate. Additional organic diluents include fats, oils, fatty acids, and fatty alcohols.

Further materials suitable for the personal care and health care are well known to the person skilled in the art and are described in many text books as well as other publications.

The general level of elastomer (E) in the cosmetic compositions may vary from 0.1% to 95% by weight, alternatively from 0.2% to 50%, alternatively from 0.5% to 25%, relative to the total weight of the cosmetic composition. The cosmetic ingredient is present at a level of from 0.01% to 99.99% by weight, relative to the total weight of the cosmetic composition. The cosmetic ingredient may be a mixture of cosmetic ingredients as listed above.

The cosmetic composition may be prepared by a process comprising the steps of

a. mixing a silicone organic elastomer comprising an amino functional group
b. and at least one cosmetic ingredient,
c. optionally in the presence of a cosmetically acceptable medium.

The cosmetic compositions may be prepared by mixing the silicone organic elastomer comprising an amino functional group with the appropriate phase ingredients, typically the oil phase, and optionally provide for a second phase, typically an aqueous or polar phase, and mix both phases together, optionally under heating.

The process may be conducted at temperatures ranging of from 15 to 90° C., alternatively of from 20 to 60° C., alternatively at room temperature (25° C.), using simple propeller mixers, counter-rotating mixers, or homogenizing mixers. No special equipment or processing conditions are typically required. Depending on the type of composition prepared, the method of preparation will be different, but such methods are well known in the art.

The cosmetic compositions may be in the form of a cream, a gel, a powder (free flowing powder or pressed), a paste, a solid, freely pourable liquid, an aerosol. The cosmetic compositions may be in the form of monophasic systems, biphasic or alternate multi phasic systems; emulsions, e.g. oil-in-water, water-in-oil, silicone-in-water, water-in-silicone; multiple emulsions, e.g. oil-in-water-in-oil, polyol-in-silicone-in-water, oil-in-water-in-silicone.

Skin care compositions include shower gels, soaps, hydrogels, creams, lotions and balms; antiperspirants; deodorants such as sticks, soft solid, roll on, aerosol, and pump sprays; skin creams; skin care lotions; moisturizers; facial treatments such as wrinkle control or diminishment treatments; exfoliates; body and facial cleansers; bath oils;

perfumes; colognes; sachets; sunscreens; mousses; patches; pre-shave and after-shave lotions; shaving soaps; shaving lathers; depilatories; make-ups; color cosmetics; foundations; concealers; blushes; lipsticks; eyeliners; mascaras; oil removers; color cosmetic removers, powders, and kits thereof.

Hair care compositions include shampoos, rinse-off conditioners, leave-in conditioners and styling aids, gels, sprays, pomades, mousses, waxes, cuticle coats, hair colorants, hair relaxants, hair straighteners, permanents, and kits thereof.

Nail care compositions include color coats, base coats, nail hardeners, and kits thereof.

Health care compositions may be in the form of ointments, creams, gels, mousses, pastes, patches, spray on bandages, foams and/or aerosols or the like, medicament creams, pastes or sprays including anti-acne, dental hygienic, antibiotic, healing promotive, which may be preventative and/or therapeutic medicaments, and kits thereof.

The cosmetic compositions may be used by the standard methods, such as applying them to the human or animal body, e.g. skin or hair, using applicators, brushes, applying by hand, pouring them and/or possibly rubbing or massaging the composition onto or into the body. Removal methods, for example for colour cosmetics are also well known standard methods, including washing, wiping, peeling and the like.

The cosmetic compositions are applied topically to the desired area of the skin or hair in an amount sufficient to provide a satisfactory cleansing or conditioning of the skin or hair. The compositions may be diluted with water prior to, during, or after topical application, and then subsequently rinsed or wiped off of the applied surface, for example rinsed off of the applied surface using water or a water-insoluble substrate in combination with water.

The invention also comprises a method of caring for keratinous substrates, such as hair or skin, by applying to it a cosmetic composition according to the first aspect of the invention.

The method to care for keratinous substrates comprises the steps of
a. Providing for a cosmetic composition comprising a silicone organic elastomer comprising an amino functional group, and at least one cosmetic ingredient, optionally in a cosmetically acceptable medium,
b. Applying the composition to the keratinous substrate
c. Optionally rinsing.

Once applied to the keratinous substrate, the cosmetic composition may be left to stand. The standing time may range of from 1 second to 24 hours or more, alternatively, from 1 second to 12 hours, alternatively from 1 second to 60 minutes. The standing time may optionally be followed by the optional rinsing step.

The cosmetic compositions may be used on hair in a conventional manner. An effective amount of the composition for washing or conditioning hair is applied to the hair. Such effective amounts generally range from about 1 g to about 50 g, preferably from about 1 g to about 20 g. Application to the hair typically includes working the cosmetic composition through the hair such that most or all of the hair is contacted with the cosmetic composition. These steps can be repeated as many times as desired to achieve the desired benefit.

Benefits obtained from using the cosmetic compositions on hair include one or more of the following benefits: color retention, improvement in coloration process, hair conditioning, softness, detangling ease, silicone deposition, anti-static, anti-frizz, lubricity, shine, strengthening, viscosity, tactile, wet combing, dry combing, straightening, heat protection, styling, or curl retention.

The cosmetic compositions may be used on skin in a conventional manner. An effective amount of the composition for the purpose is applied to the skin. Such effective amounts generally range from about 1 mg/cm2 to about 3 mg/cm2. Application to the skin typically includes working the cosmetic composition into the skin. This method for applying to the skin comprises the steps of contacting the skin with the cosmetic composition in an effective amount and then rubbing the composition into the skin. These steps can be repeated as many times as desired to achieve the desired benefit.

Benefits obtained from using the cosmetic compositions on skin include one or more of the following benefits: skin softness, suppleness, moisturisation, skin feel, foam generation, durability, substantivity, long lasting, long wear, color enhancement.

The cosmetic composition may be used to care for keratinous substrates, that is, to cleanse, to condition, to refresh, to make up, to remove make up, to fix hair.

### Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention. All percentages are in wt. %. All measurements were conducted at 23° C. unless indicated otherwise.

### Preparation of XZ′ₙ Derivative 1:

Amination epoxide reaction using Allyl Glycidyl Ether (AGE) and 1-Hexadecylamine: 80.79 g of 1-hexadecylamine (CH₃(CH₂)₁₄CH₂NH₂, mw=241.46 g/mol, Aldrich, 98%) and 100 g of isopropanol were mixed under heating at 50° C. to melt the 1-hexadecylamine (melting point is ˜35° C.), then only 76.42 g of allyl glycidyl ether (C₆H₁₀O₂, bp=154° C., mw=114 g/mol, Aldrich, >99%) was added in several aliquots. This represented a 100% excess of AGE. The mixture was held at 60° C. overnight. The volatiles (isopropanol and excess AGE) were removed the following day via vacuum stripping. The reaction product was examined by 13C NMR for unreacted amine. The resulting product is C₂₈H₅₅NO₄, 469.75 g/mol.

Preparation of a Silicone Organic Elastomer Comprising an Amino Functional Group

### Elastomer 1:

36.11 g (29.02 mmol) of an organohydrogensiloxane with an average formula of Me₃SiO(Me₂SiO)ₓ(MeHSiO)_{y}SiMe₃, where x and y are of a value such that the organohydrogensiloxane has a viscosity of 107 mm²/s (cSt) at 23° C. and contains 0.0810 wt. % H as Si—H, 3.94 g of XZ′ₙ derivative 1, 10.94 g of a polyalkylene oxide with an average structure of, CH₂═C(CH₃)CH₂O(CH₂CH(CH₃)O)₂₀CH₂(CH₃)CH═CH₂ and 249 g of isododecane were mixed. Next, 0.52 g of a 1% solution of platinum IV was added. The jar was sealed and heated to 70° C. using a water bath and stirring continued until the reaction mixture gelled. The solution was observed to gel in 30 minutes and was held at 70° C. for three hours to complete the addition cure. The elastomer gels, as made in preparation of elastomer above, were made into gel pastes using a high shear mixing. The shear steps include the addition of additional carrier fluid (solvent) and organovinylsiloxane. The materials were sheared in a Waring Commercial Laboratory Blender. In shear step 1, the gel was sheared for 20 seconds at setting 1, then 20 seconds at setting 3, then 20 seconds at setting 5. Solvent and organovinylsiloxane were added followed by shearing for 30 seconds at each of the following settings: 1, 2, 3, 3. Between each setting, the material was scraped from the sides of the mixer cup using a spatula.

### Cosmetic Compositions

Non Transfer Lipstick—Table 1, prepared as follows:
1. Mix Phase A in Dental Mixer until uniform color. Add Elastomer 1 to phase A and mix on dental mixer until homogenous
2. Place Lanolin Oil, Avocado oil, ceresin wax, candelilla wax, Octyldodecaneol, AMS-C30 Cosmetic wax, and oleyl alcohol in beaker and heat until 80° C. with gentle mixing until melted
3. Add Elastomer 1 and phase A to beaker, mix phase A cup with FZ-3196 (dental mixer) and pour into beaker, rinse phase A cup with 2 cSt fld (dental mixer) and pour into beaker,
4. Homogenize
5. Pour into mold and allow to cool

TABLE 1

| | |
|---|---|
| Ingredient | % wt |

Phase A

| | |
|---|---|
| Caprylyl Methicone | 6.25 |
| Alkyl Silane Treated Iron Oxide | 11.75 |
| Alkyl Silane Treated Sericite | 3.50 |

Phase B

| | |
|---|---|
| Elastomer 1 | 12.50 |
| Lanolin Oil | 11.25 |
| Persea Gratissima (Avocado) Oil | 2.50 |
| Ceresin | 5.00 |
| Candelilla Wax | 13.75 |
| Octyldodecaneol | 16.25 |
| C30-45 Alkyl Methicone (and) C30-45 Olefin | 7.25 |
| Oleyl Alcohol | 10.00 |

Lip gloss—Table 2, prepared as follows:
1. Add ingredients in phase A one by one in order under mixing
2. Mix phase A well to 75° C.
3. Add phase B into phase A with mixing
4. Mix phases (A+B) well
5.

TABLE 2

| | |
|---|---|
| Ingredient | % wt |

Phase A

| | |
|---|---|
| Dimethicone | 9.87 |
| Cyclopentasiloxane (and) Dimethiconol | 39.90 |
| Phenyltrimethicone | 6.00 |
| Bis-hydroxyethoxypropyl Dimethicone | 12.00 |
| Dimethicone (and) Trimethylsiloxysilicate | 8.00 |
| Olea Europaea (Olive) Fruit Extract | 2.00 |
| Ethylhexyl Salicylate (Octyl Salicylate) | 6.00 |
| Elastomer 1 | 15.23 |

Phase B

| | |
|---|---|
| Silica Silylate | 1.00 |

Sun Cream—Table 3, prepared as follows:
1. Mix phase A ingredients together until all ingredients are completely dissolved
2. Mix phase B ingredients together
3. Add phase B to phase A with gentle mixing

TABLE 3

| | |
|---|---|
| Ingredient | % wt |

Phase A

| | |
|---|---|
| Trimethylsiloxysilicate (and) Polypropylsilsesquioxane | 1.00 |
| Caprylyl Methicone | 5.00 |
| Dimethicone | 4.00 |
| Dimethicone | 1.08 |
| Elastomer 1 | 3.92 |
| Ethylhexyl Methoxycinnamate | 7.50 |
| PEG-10 Dimethicone | 2.00 |
| Titanium Dioxide (and) Isohexadecane (and) Triethylhexanoin | 14.00 |
| (and) Aluminium Stearate (and) Polyhydroxystearic Acid (and) | |
| Alumina | |
| Alcohol | 1.00 |

Phase B

| | |
|---|---|
| Sodium Chloride | 1.00 |
| Glycerin | 5.00 |
| Water | 54.50 |

Antiperspirant Gel—Table 4, prepared as follows:
1. Mix phase A well
2. Dissolve aluminum chloride in mixture of water and propylene glycol, with mixing
3. Drop phase B into Phase A slowly
4. Mix final gel well.

TABLE 4

| | |
|---|---|
| Ingredient | % wt |

Phase A

| | |
|---|---|
| Cyclopentasiloxane (and) PEG-12 Dimethicone Crosspolymer | 10.00 |
| Cyclopentasiloxane | 6.15 |
| Elastomer 1 | 13.85 |

Phase B

| | |
|---|---|
| Aluminum Zirconium Tetrachlorohydrate GLY | 25.00 |
| Propylene Glycol | 7.00 |
| Water | 38.00 |

Antiperspirant Soft Solid—Table 5, prepared as follows:
1. Slowly add ingredient 2 to ingredient 1 while mixing until homogeneous
2. Slowly add ingredient 3 with mixing
3. Continue mixing until completely dispersed, which may take up to 1-2 hours depending on batch size
4. Add ingredient 4 with mixing
5. Heat to 60° C.
6. Mix gently until melted
7. Add ingredient 5 with mixing
8. Maintain temperature at 60° C.
9. Stir until mixture is homogeneous
10. Cool to 50° C.
11. Pour into containers

TABLE 5

| | Ingredient | % wt |
|---|---|---|
| 1 | Elastomer 1 | 5.00 |
| 2 | Cyclopentasiloxane | 69.50 |
| 3 | Silica | 0.50 |
| 4 | Stearyl Alcohol | 5.00 |
| 5 | Aluminum Zirconium Tetrachlorohydrex GLY | 20.00 |

Body Care Lotion—Table 6, prepared as follows:
1. Separately mix phases A and B, well
2. Slowly drip phase B into phase A (RPM 1376)
3. Mix phases (A+B) well for 15 minutes

TABLE 6

| | |
|---|---|
| Ingredient | % wt |

Phase A

| | |
|---|---|
| PEG/PPG-19/19 Dimethicone (and) C13-16 Isoparaffin (and) C10- | 4.20 |
| 13 Isoparaffin | |
| Elastomer 1 | 21.88 |
| Glycine Soja (Soybean) Oil | 5.30 |
| Phenyl Trimethicone (and) Dimethiconol | 2.10 |
| Cyclopentasiloxane | 2.10 |
| Caprylyl Methicone | 4.20 |
| Cyclopentasiloxane (and) Polypropylsilsesquioxane | 2.10 |

Phase B

| | |
|---|---|
| Water | 51.82 |
| Glycerin | 6.30 |

Body Cream Natural Butter—Table 7, prepared as follows:
1. Add phase A ingredients to a mixing vessel and heat to 85° C. using a water while mixing at 400 RPM with a marine propeller type blade
2. Heat phase B to 85° C.
3. Increase mixing speed of phase A to 600 RPM
4. Add phase B to phase A, mixing until homogenous
5. Remove from heat. Cool with mixing to 50° C.
6. Add phase C
7. Continue mixing and cool to 35° C.
8. Pour into an appropriate container

TABLE 7

| | |
|---|---|
| Ingredient | % wt |

Phase A

| | |
|---|---|
| Elastomer 1 | 17.00 |
| C30-45 Alkyldimethylsilyl Polypropylsilsesquioxane | 3.90 |
| Butyrospermum Parkii (Shea) Butter | 9.50 |
| Garcinia Indica Seed Butter | 1.00 |
| Mangifera Indica (Mango) Seed Butter | 2.00 |
| Behentrimonium Methosulfate and Cetearyl Alcohol | 2.00 |
| Cetearyl Alcohol and Polysorbate 60 | 6.00 |
| Dimethicone | 2.00 |
| Caprylyl Methicone | 2.00 |
| Stearyl Dimethicone | 1.50 |
| Dimethicone (and) Dimethiconol | 2.50 |

Phase B

| | |
|---|---|
| Water | 49.85 |

Phase C

| | |
|---|---|
| Phenoxyethanol (and) Methylparaben (and) Isopropylparaben | 0.75 |
| (and) Isobutylparaben | |

Light Feel O/W Lotion—Table 8, prepared as follows:
1. Mix water and sodium polyacrylate dispersion until homogeneous
2. Add the remaining ingredients of Phase B, then mix until uniform at 1000 RPM using a marine propeller
3. Add Elastomer 1 to a separate mixing vessel
4. Add each of the Phase A ingredients to the Elastomer 1 one at a time, mixing until uniform with each addition
5. Add phase A to phase B with good mixing

TABLE 8

| | |
|---|---|
| Ingredient | % wt |

Phase A

| | |
|---|---|
| Elastomer 1 | 10.80 |
| Isodecyl Neopentanoate | 1.40 |
| Cetyl Dimethicone | 1.40 |
| Caprylyl Methicone | 3.60 |
| Dicaprylyl Ether | 3.60 |

Phase B

| | |
|---|---|
| Water | 69.50 |
| Sodium Polyacrylate (and) Dimethicone (and) Cyclopentasiloxane | 4.00 |
| (and) Trideceth-6 (and) PEG/PPG-18/18 Dimethicone | |
| Rosmarinus Officinalis (Rosemary) Leaf Extract | 5.10 |
| Dehycroacetic acid (and) Benzyl alcohol | 0.60 |

Hair Shampoo—Table 9, prepared as follows:
1. Add Ingr.1 to Ingr.2 and heat to 40° C. and begin mixing at 400 rpm.
2. Add Ingr.5 and heat to 70° C.
3. Add Ingr.3 and Ingr.6 and heat to 76° C. Once Ingr.6 is completely melted, remove heat and cool to 40° C.
4. Adjust mixing speed to 300 rpm and add Ingr.4, Ingr.7, Ingr.8 and compensate for the water loss.
5. Continue mixing for 15 minutes.

TABLE 9

| | |
|---|---|
| Ingredient | % wt |

| | | |
|---|---|---|
| 1 | Water | 56.50 |
| 2 | Sodium Lauryl Ether Sulfate (SLES) | 30.00 |
| 3 | Cocamide DEA | 3.00 |
| 4 | Cocamidopropyl Betaine | 7.00 |
| 5 | Polyquaternium-10 | 0.30 |
| 6 | PEG-150 Pentaerythryl Tetrastearate | 1.00 |
| 7 | Elastomer 1 | 2.00 |
| 8 | DMDM Hydantoin | 0.20 |

Hair Conditioner 1—Table 10, prepared as follows:
1. Ingr.1 is added to the mixing vessel and heated to 75° C.
2. With moderate mixing, Ingr.2 is dispersed until fully dissolved.
3. Heat is decreased to 60° C. and Ingr.3 and Ingr.4 are added.
4. Heat is then decreased to 40° and Ingr.5 is added to the base conditioner. The conditioner is mixed for 5-10 minutes and then Ingr.6 is added.
5. The water loss is compensated for and the formulation is mixed for an additional 5 minutes.
6. The final pH of the conditioner formulations are all approximately 6-7.

TABLE 10

| | |
|---|---|
| Ingredient | % wt |

| | | |
|---|---|---|
| 1 | Water | 94.30 |
| 2 | Hydroxyethylcellulose | 1.50 |
| 3 | Cetearyl Alcohol | 1.00 |
| 4 | PEG-100 Stearate & Glyceryl Stearate | 1.00 |
| 5 | Elastomer 1 | 2.00 |
| 6 | DMDM Hydantoin | 0.20 |

Hair Conditioner 2—Table 11, prepared as follows:
1. Mix ingredients of phase A together
2. Add phase B to phase A with mixing
3. Mix Phase C together and add to Phase (A+B) with mixing

TABLE 11

| | |
|---|---|
| Ingredient | % wt |

Phase A

| | |
|---|---|
| Water | 90.60 |
| Propylene Glycol (and) Diazolidinyl Urea (and) Iodopropynyl | 0.10 |
| Butylcarbamate | |
| Xanthan Gum | 0.20 |

Phase B

| | |
|---|---|
| Polyquaternium-10 | 0.10 |

Phase C

| | |
|---|---|
| Elastomer 1 | 4.00 |
| Cyclopentasiloxane (and) Dimethiconol | 2.50 |
| Polyacrylamide (and) C13-14 Isoparaffin (and) Laureth-7 | 2.50 |

Foundation—Table 12, prepared as follows:
1. Mix ingredients of phase A together.
2. Add phase B to phase A with mixing.
3. Mix Phase C ingredients together and add to Phase (A+B) with mixing.

TABLE 12

| | |
|---|---|
| Ingredient | % wt |

Phase A

| | |
|---|---|
| Dimethicone and PEG/PPG-18/18 Dimethicone | 7.50 |
| Iron Oxides and Triethoxycaprylylsilane | 3.50 |
| Iron Oxides and Triethoxycaprylylsilane | 2.18 |
| Iron Oxides and Triethoxycaprylylsilane | 1.37 |
| Titanium Dioxide and Triethoxycaprylylsilane | 2.50 |
| Caprylyl Methicone | 4.00 |
| Dimethicone | 5.56 |

Phase B

| | |
|---|---|
| Elastomer 1 | 15.50 |

Phase C

| | |
|---|---|
| Sodium Chloride | 1.00 |
| Glycerin | 3.00 |
| Water | 53.89 |

Hair color—Table 13, prepared as follows:
1. Mix ingredients of phase A together.
2. Add phase B to phase A with mixing.
3. Mix Phase C ingredients together and add to Phase (A+B) with mixing.

TABLE 13

| Ingredients | Wt % |
|---|---|
| Cetyl Alcohol | 2.50 |
| Stearyl Alcohol | 2.50 |
| Oleth-30 | 3.60 |
| Oleic Acid | 2.70 |
| Oleyl-Cetyl Alcohol 80/85 | 2.50 |
| Ethyleneglycol Monostearate | 1.00 |
| Polyquaternium 7 | 3.00 |
| DTPA Na5 (40%) | 1.80 |
| Sodium Metabisulfite | 0.60 |
| Sodium Erythorbate | 0.20 |
| Monoethanolamine (99%) | 0.66 |
| Propylene Glycol | 5.00 |
| Acrylates/C10-30 Alkyl Acrylate Cross-Polymer | 0.30 |
| Ammonium Hydroxide (27%) | 10.02 |
| Colouring Composition comprising phenylene | 0.82 |
| diamine, resorcinol, p-aminophenol, m-aminophenol, | |
| 2,4-diaminophenol sulfate | |
| Elastomer 1 | 2.00 |
| Water | q.s. to 100 |

1. A cosmetic composition comprising:
a silicone organic elastomer comprising an amino functional group; and
at least one cosmetic ingredient,
optionally in a cosmetically acceptable medium;
wherein the silicone organic elastomer is the reaction product of;
(A) a linear, branched or cyclic organohydrogensiloxane comprising at least one silicon-bonded hydrogen atom, and
(B) a XZ′ₙ derivative comprising at least two unsaturated aliphatic groups,
where X is an amine group containing compound, Z′ is a ring-opened ethylenically-unsaturated epoxide comprising at least one unsaturated aliphatic group and n=1 or 2,
in the presence of
(C) a hydrosilylation catalyst.

2. The cosmetic composition of claim 1, wherein the cosmetically acceptable medium is present.

3. The cosmetic composition of claim 1, wherein the cosmetic ingredient is selected from emollients, waxes, film formers, moisturizers, surface active materials such as surfactants or detergents or emulsifiers, thickeners, water phase stabilizing agents, pH controlling agents, preservatives and cosmetic biocides, sebum absorbants or sebum control agents, vegetable or botanical extracts, vitamins, proteins or amino-acids and their derivatives, pigments, colorants, fillers, silicone conditioning agents, cationic conditioning agents, hydrophobic conditioning agents, UV absorbers, sunscreen agents, antidandruff agents, antiperspirant agents, deodorant agents, skin protectants, hair dyes, nail care ingredients, fragrances or perfume, antioxidants, oxidizing agents, reducing agents, propellant gases, fatty alcohols, colour care additives, anticellulites, pearlising agents, chelating agents, styling agents, ceramides, suspending agents and mixtures thereof.

4. The cosmetic composition of claim 1, which is in the form of a cream, a gel, a powder, a paste, a solid, a freely pourable liquid, or an aerosol.

5. The cosmetic composition of claim 4, further defined as a skin care composition.

6. The cosmetic composition of claim 4, further defined as a hair care composition

7. A process for preparing a cosmetic composition comprising the step of
mixing a silicone organic elastomer comprising an amino functional group
and at least one cosmetic ingredient,
optionally in the presence of a cosmetically acceptable medium.

8. The process according to claim wherein the cosmetic composition is prepared by mixing the silicone organic elastomer with one or more phase ingredients, and optionally provide for a second phase, and mix both phases together, optionally under heating.

9. A method to care for keratinous substrates comprising the steps of:
providing a cosmetic composition comprising a silicone organic elastomer comprising an amino functional group, and at least one cosmetic ingredient, optionally in a cosmetically acceptable medium;
applying the composition to the keratinous substrate; and
optionally rinsing the keratinous substrate.

10. The method according to claim 9, wherein the keratinous substrate is skin, hair, or a combination thereof.

11. The cosmetic composition of 3, which is in the form of a cream, a gel, a powder, a paste, a solid, a freely pourable liquid, or an aerosol.

12. The process according to claim 8, wherein the phases are an oil phase and an aqueous or polar phase.

13. The cosmetic composition of claim 1, wherein the reaction product is also formed in the presence of a carrier fluid.

14. The cosmetic composition of claim 1, wherein component (A) comprises an organohydrogensiloxane with the average formula
Me₃SiO(Me₂SiO)ₓ(MeHSiO)_{y}SiMe₃
where Me is CH₃ and x and y are each of a value such that the organohydrogensiloxane has a viscosity of 107 mm²/s (cSt) at 23° C. and contains 0.0810 wt. % H as Si—H.

15. The cosmetic composition of claim 1, wherein component (B) comprises the reaction product of 1-hexadecylamine and allyl glycidyl ether (AGE).

16. The cosmetic composition of claim 1, wherein the silicone organic elastomer is the further reaction product of (B1) a crosslinker.

17. The cosmetic composition of claim 16, wherein component (B1) is selected from:
an alpha, omega-diene;
a polyoxyalkylene comprising two unsaturated aliphatic groups;
a glycerol ether comprising two unsaturated aliphatic groups; or
a siloxane polymer comprising two unsaturated aliphatic groups.

18. The cosmetic composition of claim 16, wherein component (B1) comprises a polyoxyalkylene of the average formula CH₂═C(CH₃)CH₂O(CH₂CH(CH₃)O)₂₀CH₂(CH₃)CH═CH₂.

19. The cosmetic composition of claim 1, wherein the silicone organic elastomer is the further reaction product of (D) a compound having one unsaturated aliphatic hydrocarbon group.

20. The cosmetic composition of claim 19, wherein component (D) is selected from:
(D¹) a hydrocarbon containing 6-30 carbons and having one unsaturated aliphatic group;
(D²) a polyoxyalkylene having one unsaturated aliphatic group;
(D³) a XZ′ₙ derivative having one unsaturated aliphatic group;
(D⁴) a linear or branched siloxane polymer comprising one unsaturated aliphatic group;
(D⁵) a polyol component comprising one unsaturated aliphatic group; or mixtures thereof.
